(19) United States
(12) Patent Application Publication
(10) Pub. No.: US 2010/0113599 A1
(43) Pub. Date: May 6, 2010

(54) AMPHIPATHIC CARBOXYLATES FOR THE TREATMENT OF IMMUNE-RELATED DISORDERS

(75) Inventor: Jacob Bar-Tana, Jerusalem (IL)

Correspondence Address: BUCHANAN, INGERSOLL & ROONEY PC, POST OFFICE BOX 1404, ALEXANDRIA, VA, 22313-1404, US

(73) Assignee: Syndromex Ltd. c/o Yissum Research Development Company, of, Jerusalem (IL)

(21) Appl. No.: 12/528,706

(22) PCT Filed: Feb. 26, 2008
(86) PCT No.: PCT/IL2008/000245

§ 371 Date: Jan. 13, 2010

(30) Foreign Application Priority Data
Feb. 26, 2007 (IL) 181577

(51) Int. Cl. A61K 31/194; A61P 37/02; A61P 37/00; A61P 29/00; A61P 35/00; A61P 25/28; A61P 9/00; C07C 55/20

(52) U.S. Cl. 514/574; 562/590

(57) ABSTRACT

The present invention relates to the use of at least one long-chain optionally-substituted amphipatic carboxylates (known as MEDICA drugs) or any salt, ester or amid thereof or any combination or mixture thereof, for the treatment and prevention of immune-related disorders, particularly, inflammatory conditions. More specifically, the invention relates to particular analogs of these MEDICA drugs, the 3,3,14,14 tetramethyl-hexadecanedioic acid (M16ββ), 4,4,15,15 tetramethyl-octadecanedioic acid (M18γγ) and 2,2,15,15-tetramethyl-hexadecanedioic acid (M16αα). The invention further provides methods, oral compositions and kits for the treatment and prevention of immune-related disorders using these MEDICA drugs. The disease is any one of multiple sclerosis, neurodegenerative disease, an atherosclerotic disease, inflammatory bowel diseases, arthritis. Wherein said atheroslerotic disease is any one of cardiovascular disease, cerebrovascular disease and peripheral vessel disease, and a malignant proliferative disorder line carcinoma, myeloma, leukemia, lymphoma, sarcoma and melanoma.

### FIELD OF THE INVENTION

The present invention relates to the use of long-chain optionally substituted amphipatic carboxylates, for the treatment and prevention of immune-related disorders, particularly, inflammatory conditions. The invention further provides methods of treatment of such disorders using these MEDICA drugs.

### BACKGROUND OF THE INVENTION

Throughout this application, various publications, including United States patents, are referenced by author and year and patents by number. The disclosures of these publications and patents and patent applications in their entireties are hereby incorporated by reference into this application in order to more fully describe the state of the art to which this invention pertains.

Substituted long chain dicarboxylic acids (also referred to as MEDICA drugs), and in particular their 3,3,14,14-tetramethyl-hexadecanedioic acid (M16ββ), 2,2,15,15-tetramethyl-hexadecanedioic acid (M16αα) and 4,4,15,15-tetramethyl-octadecanedioic acid (M18γγ) representatives, were previously shown by the present inventors to lower triglycerides, increase HDL-C and increase sensitization to insulin, and therefore offer a treatment mode of choice for dyslipidemic patients, specifically Metabolic Syndrome CVD patients. MEDICA drugs consist of chemical entities targeting transcription factors (HNF-4α, FOXO) and protein kinases (AMPK, PKA) involved in modulating the production and clearance of plasma lipoproteins. M16ββ as representative of MEDICA drugs is effective in lowering plasma triglycerides while increasing HDL-C and sensitivity to insulin with amelioration of diabetes type 2 in animal models and in humans.

Cardiovascular diseases (CVD) are the first leading causes of death of men and women in the Western world, claiming more lives each year than the combined next four leading causes of death. Increased LDL-cholesterol (LDL-C), hypertriglyceridemia and low HDL-cholesterol (HDL-C) are major CVD risk factors. Another crucial risk factor in Metabolic Syndrome CVD patients, are inflammatory responses as reflected by the inflammatory markers (e.g., C-reactive protein (CRP), Serum amyloid A (SAA), Intercellular Adhesion Molecule 1 (ICAM1)) that promote atherosclerotic CVD. As surprisingly shown by the present invention, beyond their hypotriglyceridemic activity, MEDICA drugs targets such inflammatory markers, due to their efficacy in suppressing the transduction pathways (e.g., STAT3, NFkβ) triggered by inflammatory cytokines and leading to the expression of inflammatory products (e.g., CRP, SAA, fibrinogen, COX2, ICAM).

The applicability of immunomodulatory effect of MEDICA drugs in treating different immune-related disorders have been demonstrated by the present invention.

Therefore, one object of the invention is to provide compositions and methods for the treatment of immune-related disorders, particularly, inflammatory disorders.

These and other objects of the invention will become apparent as the description proceeds.

### SUMMARY OF THE INVENTION

In a first aspect, the invention relates to at least one long-chain optionally substituted amphipathic carboxylate or any salt, ester or amid thereof or any combination or mixture thereof, for use in the prevention or the treatment of an immune-related disorder.

According to a specific embodiment, the invention relates to particular analogs of these MEDICA drugs, the 3,3,14,14 tetramethyl-hexadecanedioic acid (M16ββ), 2,2,15,15-tetramethyl-hexadecanedioic acid (M16αα) and 4,4,15,15-tetramethyl-octadecanedioic acid (M18γγ).

Therefore, according to one embodiment, the MEDICA drugs are particularly applicable for treating and preventing immune-related conditions, for example, an inflammatory disorder, an autoimmune disease, and malignant and non-malignant proliferative disorder.

According to a second aspect, the invention relates to a method of treatment or prevention of an immune-related disorder. The method of the invention comprises the step of administering to a subject in need thereof a therapeutically effective amount of at least one xenobiotic long-chain substituted amphipathic carboxylate or any salt, ester or amid thereof or any combination or mixture thereof, or of any composition comprising the same.

According to a third aspect, the invention relates to the use of a therapeutically effective amount of at least one xenobiotic long-chain substituted amphipathic carboxylate or any salt, ester or amid thereof or any combination or mixture thereof, in the preparation of a medicament for the treatment or prevention of an immune-related disorder.

According to another embodiment, the present invention further provides an oral pharmaceutical composition comprising a therapeutically effective amount of at least one xenobiotic long-chain substituted amphipathic carboxylate and optionally at least one additional therapeutic agent, with a pharmaceutically acceptable carrier.

These and other aspects of the invention will become apparent by the hand of the following figures.

### BRIEF DESCRIPTION OF THE FIGURES

FIG. 1

Schematic illustration of the cytokine/gp130-induced expression of Acute Phase Proteins (APP).

FIG. 2

MEDICA drugs reduce Fibrinogen α expression.

HepG2 cells were incubated for 40 h with 200 mM M16αα. IL-6 (20 ng/ml) was added for the last 15 h. Total RNA was prepared and was analyzed by real time RT-PCR for fibrinogen a mRNA. Transcript levels were normalized by GAPDH.

FIG. 3

MEDICA drugs reduce SAA1 expression.

HepG2 cells were incubated for 40 h with 200 mM M16αα. IL-6 (20 ng/ml) was added for the last 15 h. Total RNA was prepared and was analyzed by real time RT-PCR for SAA1 mRNA. Transcript levels were normalized by GAPDH.

FIG. 4

MEDICA drugs reduce CRP expression.

Hep3B cells were treated with 200 mM M16αα for 24 h with or without IL-6(10 ng/ml)+IL-1(1 ng/ml). Total RNA was prepared and analyzed by real time RT-PCR for CRP mRNA. Transcript levels were normalized by β-actin.

FIG. 5

MEDICA drugs inhibit LIF-induced STAT3 phosphorylation. HepG2 cells were incubated with M16αα (200 μM) for 17 and 24 h. At each time point LIF (20 ng/ml) was added for the last 20 minutes (for protein) or for the last 3 h (for mRNA). Western blots of cell lysates were reacted with anti gp130 and anti P-STAT3 (Y705) antibodies and normalized by β-actin.

FIG. 6

MEDICA drugs inhibit IL-6-induced STAT3 phosphorylation. HepG2 cells were incubated for 40 h with 200 mM M16αα. IL-6 (20 ng/ml) was added for the last 15 h. Western blots of cell lysates were reacted, with anti GP-130, anti STAT3, anti P-STAT3 (Y705) and anti P-STAT3 (S727) antibodies and normalized by β-actin. Abbreviations: cont. (control), dim. (dimer), Tot. (total), mono. (monomer).

FIG. 7

MEDICA drugs inhibit IL-6-induced STAT3 phosphorylation in kidney cells.

Hek293 cells were incubated for 9 h with M16αα (250 μM) in serum free medium containing 0.2% Albumin. IL-6 (20 ng/ml) was added during the last 5.5 h period. Western blots of cell lysates were reacted with anti gp130 and anti P-STAT3 (Y705) antibodies and normalized by β-actin. Abbreviations: cont. (control).

FIG. 8

MEDICA drugs reduce gp130 at the protein levels.

Hek293 cells were transfected with an expression vector for gp130. Following 20 h M16αα (200 mM) was added for 9 h. Western blots of cell lysates were reacted with anti gp130 antibodies and normalized by β-actin. Abbreviations: cont. (control).

FIG. 9A-9C

M16αα inhibits HT29 colon cancer cell growth.

FIG. 9A. shows BrdU incorporation assay performed in HT29 cells treated with M16ααα200 μM as compared to untreated control (cont.).

FIG. 9B. shows Relative cell number based on trypan blue assay, of cells treated with M16αα 50, 100 and 200 μM as indicated.

FIG. 9C. shows plates of the HT29 cells treated with different concentrations of the M16αα. Abbreviations: D. (days) cont, (control), RCN (relative cell number).

FIG. 10A-10B

Growth Suppression by M16αα of Multiple Myeloma (MM) cells. MM cells were incubated in RPMI 1640 medium in the presence of added M16αα as indicated. Cell number was determined daily by Trypan Blue exclusion.

FIG. 10A. shows U266 cells;

FIG. 10B. shows RPM18226 cells.

Abbreviations: ce. Perc. (cell percentage), cont. (control)

FIG. 11A-11C

STAT3 Suppression and Induction of apoptosis by M16αα in MM U266.

U266 cells were cultured with 200 μM M16αα for time periods as indicated, lysed, Western blotted and reacted with anti STAT3, p-STAT3 (Y705), PARP or Bcl-xL antibodies.

FIG. 11A. STAT3; p-STAT3 (Y705);

FIG. 11B. PARP;

FIG. 11C. Bcl-xL.

Abbreviations: int (intact), clea. (cleaved).

FIG. 12A-12C

STAT3 Suppression and Induction of apoptosis by M16αα in MM RPMI8226 cells.

RPMI8226 cells were cultured with 200 μM M16αα for time periods as indicated, lysed, Western blotted and reacted with anti STAT3, p-STAT3 (Y705), PARP or Bcl-xL antibodies.

FIG. 12A. STAT3, p-STAT3 (Y705);

FIG. 12B. PARP;

FIG. 12C. Bcl-xL and Bcl-2.

Abbreviations: int. (intact), clea. (cleaved).

FIG. 13

Cell Cycle arrest of MM U266 by M16αα.

U266 cells were cultured with 200 μM M16αα in RPMI 1640 medium with 10% FCS for 24 h, lysed, Western blotted and reacted with anti Cyclin D1, p-P53 (S15) and p-Rb as indicated.

FIG. 14

Cell Cycle arrest of MM RPMI8226 by M16αα. RPMI8226 cells were cultured with 200 μM M16αα, in RPMI 1640 medium with 10% FCS for 24 h, lysed, Western blotted and reacted with anti p-P53(S15) and p-Rb as indicated.

FIG. 15A-15D

DSS—mice were treated with M16αα, starting 2 days prior to DSS and through the 7 days of DSS administration, Dosing: 60 mg/kg/day for 5 days followed by 30 mg/Kg/day for the last 4 days. Control mice were gavaged with the 1% CMC vehicle.

FIG. 15A. shows colon length;

FIG. 15B. shows colon weight (gr);

FIG. 15C. shows diarrhea;

FIG. 15D. shows clinical score.

Abbreviations: colo. Len. (colon length), col. We. Gr. (colon weight, gram), dia. (diarrhea), clin. Sc. (clinical score), cont. (control).

FIG. 16A-16B

Histopathological Score of DSS mice treated with M16αα.

FIG. 16A. represents Histopathological Score;

FIG. 16B. histogram of the Histopathological Score.

FIG. 17

DSS mice treated, with M16αα were examined for the expression of inflammation Markers, such as the gp130 in the intestine.

Abbreviations: cont. (control).

FIG. 18

DSS mice treated with M16αα were examined for the expression of inflammation Markers, such as the STAT3 in the colon tissue.

Abbreviations: cont. (control).

FIG. 19

DSS mice treated with M16αα were examined for the expression of inflammation Markers, such as the ITB.

Abbreviations: cont. (control).

FIG. 20

MOG35-55 peptide injected mice were treated with M16αα or vehicle (1% CMC) as indicated.

All animals were evaluated daily for their clinical score as indicated in Experimental procedures.

Abbreviations: ch. (challenge), D. (day), Clin. Sc. (clinical score) gr. (group).

FIG. 21

Western blot showing suppression of STAT3 in spleen cells of M16αα-treated EAE mice.

FIG. 22A-22B

Suppression of STAT3 in spleen cells of M16αα-treated EAE mice.

FIG. 22A. Western blot showing reduction in GP130 expression in M16αα-treated EAE mice.

FIG. 22B. Histogram summarizing the Western blot results showing reduction in GP130 expression in M16αα-treated EAE mice.

Abbreviations: cont. (control).

FIG. 23

Effect of M16αα on liver Acute Phase Proteins (APP) gene expression in EAE mice. Abbreviations: cont. (control).

FIG. 24

hCRPtg were treated for 10d with M16αα (80 mg/kg/d) dissolved in 1% CMC and administered by gavage. Plasma basal CRP was determined before and at the end of treatment by ELISA.

Abbreviations: pre. Treat. (before treatment), po. Trea. (after treatment).

FIG. 25

LPS-induced CRP.

The effect of M16αα in decreasing human CRP induced by LPS injection was analyzed in hCRPtg mice treated with M16αα for 14 days and challenged with LPS.

Abbreviations: cont. (control), tre. (treated).

### DETAILED DESCRIPTION OF THE INVENTION

Medica 16, referred to henceforth as M16, 3,3,14,14-tetramethyl-hexadecane-1,16-dicarboxylic acid, is a β,β,-methyl substituted α,ω-dicarboxylic acid of sixteen carbons in chain length. M16 is prepared essentially as described in U.S. Pat. No. 4,634,795 (M16 C₂₀H₃₈O₄, M.W. 342.5). As described above, M16 was found to be a potent hypolipidemic, antidiabetogenic, and calorigenic compound well suited for the treatment and prevention of dyslipoproteinemia (combined hypercholesterolemia-hypertriglyceridemia, low HDL-cholesterol), obesity, and impaired glucose tolerance (IGT) leading to NIDDM. The hypolipidemic effect of M16 is characterized by a pronounced decrease in plasma triglycerides and cholesterol in normolipidemic animals and normalization of plasma lipids in hyperlipidemic animal models.

The hypolipidemic effect is due to a pronounced activation of clearance of plasma chylomicrons and very-low-density lipoproteins secondary to inhibition of apolipoprotein CIII synthesis and consequent disinhibition of lipoprotein lipase activity and hepatic lipase activities. Increased plasma HDL-cholesterol is observed secondary to normalization of plasma triglycerides. MEDICA 16 thus prove to be a valuable option for the treatment of combined hypercholesterolemia-hypertriglyceridemia or for treating isolated hypertriglyceridemia with low plasma HDL-cholesterol or for lowering postprandial plasma chylomicrons.

As demonstrated by the present invention, the inventors have now investigated the immunomodulatory effect of MEDICA drugs demonstrating the feasibility of using these drugs, and particularly M16αα, M16ββ or M18γγ as a medicament for the treatment and the prevention of different immune-related disorders.

Thus, in a first aspect, the invention relates to at least one xenobiotic long-chain optionally substituted amphipathic carboxylate or any salt, ester or amid thereof or any combination or mixture thereof, for use in the prevention or the treatment of an immune-related disorder.

As used herein, a xenobiotic substance (from the Greek words xenos:stranger/foreign and bios:life) is a chemical which is found in an organism but which is not normally produced or expected to be present in it. It can also cover substances which are present in much higher concentrations than are usual.

The long-chain optionally substituted amphipathic dicarboxylic acid used by the invention may be according to one embodiment, a compound of formula (I):

HOOC—CR₁R₂—CR₃R₄—CR₃R₄—CR₅R₆—Q—CR₇R₈—CR₉R₁₀—CR₁₁R₁₂—COOH (I)
wherein R₁-R₁₂ each independently represents a hydrogen atom, an unsubstituted or substituted hydrocarbyl or a lower alkoxy group; and
wherein Q represents a diradical consisting of a linear chain of 2 to 14 carbon atoms, one or more of which may be replaced by heteroatoms, said chain being optionally substituted by inert substituents, and wherein one or more of said carbon or heteroatom chain members optionally forms part of a ring structure, and pharmaceutically acceptable salts, esters, amides, anhydrides and lactones thereof. It should be noted that the invention further refers to in vivo hydrolysable functional derivatives of the carboxylic groups thereof. According to one embodiment, the heteroatom is selected from N, P, O and S.

According to another embodiment, the salt is a salt with an inorganic or organic cation, in particular alkali metal salt, alkaline earth metal salt, ammonium salt and substituted ammonium salt; said ester is a lower alkyl ester; said an amide, is a mono- and di-substituted; said anhydride, is an anhydride with a lower alkanoic acid; and/or said lactone is formed by ring closure of either or both carboxylic groups with a free hydroxy substituent (or substituents) in the molecule of formula (I).

Still further, the hydrocarbyl may be an optionally substituted alkyl, alkenyl, alkynyl, cycloalkyl, an optionally substituted aryl, or an optionally substituted aralkyl.

According to another embodiment, each of R₁-R₁₂ is a lower alkyl and Q is a straight polymethylene chain of 2 to 14 carbon atoms.

According to another preferred embodiment, the amphipatic dicarboxylic acid is a γ,γ-substituted acid in which each of R₅-R₉ is a methyl group, each of R₁-R₄ and R₉-R₁₂ is hydrogen and Q is a straight polymethylene chain of 2 to 14 carbon atoms, as denoted by formula (II):

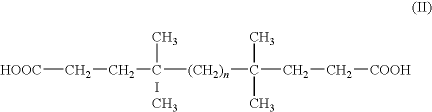

wherein n is an integer of from 2 to 14 (n=10 also referred to as M18γγ).

According to an alternative embodiment, the amphipatic dicarboxylic acid is an α,α-substituted acid wherein each of R₁, R₂, R₁₁ and R₁₂ is a methyl group, each of R₃-R₁₀ is hydrogen and Q is a straight polymethylene chain of 6 to 18 carbon atoms, as denoted by formula (III):

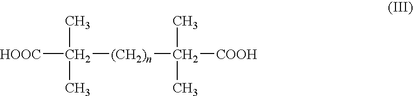

where n is an integer from 6 to 18.

According to a particular and preferred embodiment, the compound is 2,2,15,15-tetramthylhexadecane-1,16-dioic acid. This compound is referred to herein as M2001 or M16αα.

In yet another specifically preferred embodiment, the amphipatic dicarboxylic acid is a β,β-substituted acid wherein in said compound each of R₃, R₄, R₉, R₁₀ is a methyl group, each of R₁, R₂, R₅, R₅, R₇, R₈, R₁₁, R₁₂ is hydrogen and Q is a straight polymethylene chain of 4 to 16 carbon atoms, as denoted by formula (IV):

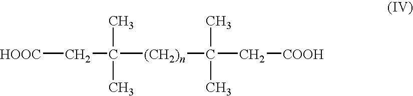

wherein n is an integer of from 4 to 16.

A particular embodiment of such compound is 3,3,14,14-tetramethylhexadecane-1,16-dioic acid, which is also referred to herein as M1001 or M16ββ.

The anti-inflammatory effect of MEDICA drugs (specifically the M16 analogs) shown in the following examples, renders these drugs alone or in a combination with an additional therapeutic agent, suitable for the treatment of immune and inflammatory conditions. Therefore, according to one embodiment, the MEDICA drugs are particularly applicable for treating and preventing immune-related conditions, for example, an inflammatory disorder, an autoimmune disease, and malignant and non-malignant proliferative disorder. By an “immune-related disorder” or “condition” is meant a pathological disease or condition associated with, caused by, linked to, usually occurring together and believed to have an impact on or by a disturbed immune system. For example, such disorder may be connected or related to dysbalance between pro-inflammatory (Th1, Th17) and anti-inflammatory (Th2) cytokines.

Therefore, in a further embodiment, the long-chain optionally substituted amphipathic dicarboxylic acid, specifically as referred herein, the MEDICA drugs, of the invention may be useful for treatment of or amelioration of inflammatory symptoms in any disease, condition or disorder where immune and/or inflammation suppression is beneficial such as, but not limited to, treatment of or amelioration of autoimmune and inflammatory symptoms in the joints, musculoskeletal and connective tissue disorders, or of autoimmune and inflammatory symptoms associated with hypersensitivity, allergic reactions, asthma, atherosclerosis, neuro-inflammatory and neurodegenerative diseases, inflammatory bowel diseases, otitis and other otorhinolaryngological diseases, dermatitis and other skin diseases, posterior and anterior uveitis, conjunctivitis, optic neuritis, scleritis, and other immune and/or inflammatory ophthalmic diseases.

According to another specific embodiment, the long-chain optionally substituted amphipathic dicarboxylic acid of the invention may be used for the treatment and prevention of an inflammatory disease such as multiple sclerosis, an atherosclerotic disease, an inflammatory bowel diseases and arthritis.

According to one specific embodiment and as exemplified by Example 5, the invention provides the long-chain (MEDICA drugs) optionally substituted amphipathic dicarboxylic acid of the invention for use in the treatment and the prevention of multiple sclerosis (MS).

Multiple sclerosis (abbreviated MS, formerly known as disseminated sclerosis or encephalomyelitis disseminata) is a chronic, inflammatory, demyelinating disease that affects the central nervous system (CNS). Disease onset usually occurs in young adults, is more common in women, and has a prevalence that ranges between 2 and 150 per 100,000 depending on the country or specific population.

MS affects the neurons in the areas of the brain and spinal cord known as the white matter. These cells carry signals in between the grey matter areas, where the processing is done, and between these and the rest of the body. More specifically, MS destroys oligodendrocytes which are the cells responsible for creating and maintaining a fatty layer, known as the myelin sheath, which helps the neurons carry electrical signals. MS results in a thinning or complete loss of myelin and, less frequently, the cutting (transection) of the neuron's extensions or axons. When the myelin is lost, the neurons can no longer effectively conduct their electrical signals. The name multiple sclerosis refers to the scars (scleroses—better known as plaques or lesions) in the white matter. Loss of myelin in these lesions causes some of the symptoms, which vary widely depending upon which signals are interrupted. However, more advanced forms of imaging are now showing that much of the damage happens outside these regions. Almost any neurological symptom can accompany the disease.

MS takes several forms, with new symptoms occurring either in discrete episodes (relapsing forms) or slowly accumulating over time (progressive forms). Most people are first diagnosed with relapsing-remitting MS but develop secondary-progressive MS (SPMS) after a number of years. Between episodes or attacks, symptoms may go away completely, but permanent neurological problems often persist, especially as the disease advances.

Although much is known about the mechanisms involved in the disease process, the cause remains elusive. The theory with the most adherents is that it results from an autoimmune reaction. The disease does not have a cure, but several therapies have proven helpful. Treatments attempt to return function after an episode, prevent new attacks, and prevent disability. As with any treatment, medications have several adverse effects, and many therapies are still under investigation.

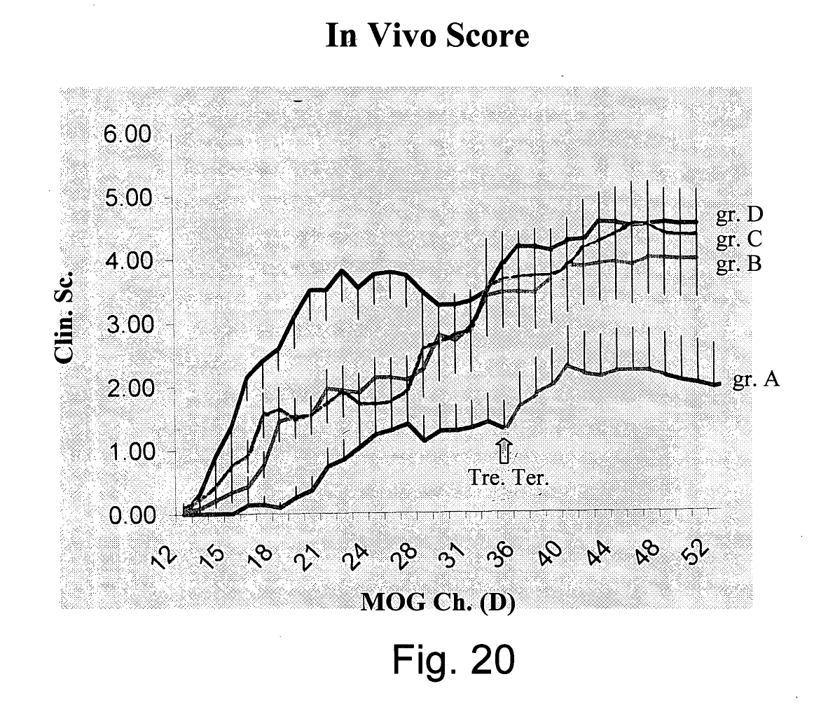

It should be specifically appreciated that the preventive effect of the MEDICA drugs demonstrated by Example 5, illustrates the feasibility of using these drugs for preventing the occurrence of further disease episodes in a patient. According to a specific embodiment, treatment, prevention or improvement in MS disease or symptoms may be reflected in improvement in clinical score (also demonstrated by FIG. 20). For example, treatment with MEDICA drugs may reduce clinical score by at least 5%, at least 10%, at least 15%, at least 20%, at least 25%, at least 30%, at least 40%, at least 50% or even by at least 55 or 60% as compared to the clinical score prior to treatment.

Still further, the MEDICA drugs of the invention are applicable in treating experimental colitis and demonstrating a significant alleviation in disease parameters, as shown by Example 4. Therefore, according to another specifically preferred embodiment, the long-chain optionally substituted amphipathic dicarboxylic acid of the invention may be used for the treatment of IBD (inflammatory bowel diseases), for example, colitis and Crohn's disease. According to a specific embodiment, treatment, prevention or improvement in colitis or in Crohn's disease may be reflected in improvement in clinical score and histo-pathological score. For example, treatment with MEDICA drugs may reduce at least one of clinical score and histo-pathological score by at least 5%, at least 10%, at least 15%, at least 20%, at least 25%, at least 30%, at least 40%, at least 50% or even by at least 55 or 60% as compared to the clinical score prior to treatment.

Inflammatory bowel diseases (IBD) are common gastrointestinal disorders that can be perceived as being the result of a dysbalance between Th1-pro-inflammatory, Th17-pro-inflammatory and Th2-anti-inflammatory subtypes of immune responses.

Crohn's disease is an ongoing disorder that causes inflammation of the digestive tract, also referred to as the gastrointestinal (GI) tract. Crohn's disease can affect any area of the GI tract, from the mouth to the anus, but it most commonly affects the lower part of the small intestine, called the ileum. The swelling extends deep into the lining of the affected organ. The swelling can cause pain and can make the intestines empty frequently, resulting in diarrhea.

As indicated above, Crohn's disease is an inflammatory bowel disease, the general name for diseases that cause swelling in the intestines. Because the symptoms of Crohn's disease are similar to other intestinal disorders, such as irritable bowel syndrome and ulcerative colitis, it can be difficult to diagnose. Ulcerative colitis causes inflammation and ulcers in the top layer of the lining of the large intestine, In Crohn's disease, all layers of the intestine may be involved, and normal healthy bowel can be found between sections of diseased bowel. Crohn's disease may also be called ileitis or enteritis.

It should be noted that the MEDICA drugs of the invention may be also applicable for the treatment or prevention of colitis. Ulcerative Colitis (U.C.) is a chronic (long lasting) inflammation of the lining of the colon (large bowel) and rectum. The lining becomes inflamed and ulcerated. The inflammation may be limited to the rectum (proctitis) or affect the whole of the colon and rectum.

The results presented in Example 6, treatment with the MEDICA drugs of the invention results in decreasing the expression of CRP (a pro-inflammatory causal agent for atherosclerosis). Therefore, according to another embodiment, the invention provides the use of these MEDICA drugs for the treatment and prevention of atherosclerosis. According to a specific embodiment, treatment, prevention or improvement in atherosclerosis may be reflected in reduction in CRP expression levels. For example, treatment with MEDICA drugs may reduce CRP expression levels by at least 5%, at least 10%, at least 15%, at least 20%, at least 25%, at least 30%, at least 40%, at least 50% or even by at least 55 or 60% as compared to the CRP expression levels prior to treatment.

Atherosclerotic condition or disease may be any one of cardiovascular disease, cerebrovascular disease and peripheral vessel disease. Atherosclerosis underlies most coronary artery disease and thus contributes to a major cause of morbidity and mortality of modern society. Atherosclerosis is a slowly progressive disease inflammatory disease of the arterial wall characterized by the accumulation of cholesterol within the arterial wall and an inflammatory response driven by sub-endothelial macrophages. The atherosclerotic process begins when LDL-C becomes trapped within the vascular wall. Oxidation of the LDL-C results in the adhesion of monocytes to the endothelial cells lining the vessel wall. These monocytes are activated and migrate into the sub-endothelial space where they are transformed into macrophages. The oxidized LDL-C is taken up through the scavenger receptor on the macrophage leading the formation of foam cells and secretion of inflammatory cytokines and chemokines. A fibrous cap is generated through the proliferation and migration of arterial smooth muscle cells, thus creating an atherosclerotic plaque. Lipids depositing in atherosclerotic legions are derived primarily from plasma apo B containing lipoproteins. These include chylomicrons, LDL-C, IDL, and VLDL. This accumulation forms bulky plaques that inhibit the flow of blood until a clot eventually forms, obstructing an artery and causing a heart attack or stroke. Therefore, high levels of total-C, LDL-C, apolipoprotein B (apo-B) and decreased levels of HDL-C are considered to promote atherosclerosis. Cardiovascular morbidity and mortality can vary directly with the level of total-C and LDL-C and inversely with the level of HDL-C.

Coronary heart disease is a multifactorial disease in which the incidence and severity are affected by the lipid profile, the presence of diabetes and the sex of the subject. Incidence is also affected by smoking and left ventricular hypertrophy which is secondary to hypertension.

In another embodiment, the long-chain optionally substituted amphipathic dicarboxylic acid, specifically, the MEDICA drugs, of the invention are useful for treatment of or amelioration of an autoimmune disease such as, but not limited to, Eaton-Lambert syndrome, Goodpasture's syndrome, Greave's disease, Guillain-Barr syndrome, autoimmune hemolytiC anemia (AIHA), hepatitis, insulin-dependent diabetes mellitus (IDDM), systemic lupus erythematosus (SLE), myasthenia gravis, plexus disorders e.g. acute brachial neuritis, polyglandular deficiency syndrome, primary biliary cirrhosis, rheumatoid arthritis, scleroderma, thrombocytopenia, thyroiditis e.g. Hashimoto's disease, Sogren's syndrome, allergic purpura, psoriasis, mixed connective tissue disease, polymyositis, dermatomyositis, vasculitis, polyarteritis nodosa, polymyalgia rheumatica, Wegener's granulomatosis, Reiter's syndrome, Behget's syndrome, ankylosing spondylitis, pemphigus, bullous pemphigoid, dermatitis herpetiformis and insulin dependent diabetes.

The immunomodulatory effect of MEDICA drugs shown by the invention has been also exemplified in models demonstrating malignant disorders (for example, Examples 2 and 3). Therefore, according to another embodiment, the long-chain optionally substituted amphipathic dicarboxylic acid of the invention, specifically, the MEDICA drugs may be used for the treatment and prevention of a malignant proliferative disorder, for example, carcinoma, myeloma, leukemia, lymphoma, sarcoma and melanoma.

More particularly, the MEDICA drugs of the invention can be used for the treatment or inhibition of non-solid cancers, e.g. hematopoietic malignancies such as all types of leukemia, e.g. acute lymphocytic leukemia (ALL), acute myelogenous leukemia (AML), chronic lymphocytic leukemia (CLL), chronic myelogenous leukemia (CML), myelodysplastic syndrome (MDS), mast cell leukemia, hairy cell leukemia, Hodgkin's disease, non-Hodgkin's lymphomas, Burkitt's lymphoma and multiple myeloma, as well as for the treatment or inhibition of solid tumors such as head and neck tumors, tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma. It should be recognized that breast cancer and prostate cancer are of specific interest.

Example 2 clearly demonstrate the beneficial effect of the MEDICA drugs on HT-29 colon carcinoma model, therefore, according to one specific embodiment, the MEDICA drugs of the invention may be used for the treatment and prevention of colon carcinoma. Colorectal cancer, also called colon cancer or bowel cancer, includes cancerous growths in the colon, rectum and appendix. It is the third most common form of cancer and the second leading cause of cancer-related death in the Western world. Colorectal cancer causes 655,000 deaths worldwide per year. Many colorectal cancers are thought to arise from adenomatous polyps in the colon. These mushroom-like growths are usually benign, but some may develop into cancer over time. The majority of the time, the diagnosis of localized colon cancer is through colonoscopy. Therapy is usually through surgery, which in many cases is followed by chemotherapy.

According to one specific embodiment, treatment or prevention of a proliferative disorder, specifically, solid tumor such as colon cancer may be reflected in reduction in tumor size. For example, treatment with MEDICA drugs may reduce tumor size and metastasis by at least 5%, at least 10%, at least 15%, at least 20%, at least 25%, at least 30%, at least 40%, at least 50% or even by at least 55 or 60% as compared to the tumor size and metastasis prior to treatment.

In yet another embodiment, the MEDICA drugs of the invention may be used for the treatment and prevention of Multiple myeloma, as exemplified by Example 3. Multiple myeloma, as used herein (also known as MM, myeloma, plasma cell myeloma, or as Kahler's disease after Otto Kahler) is a progressive hematologic disease. Multiple myeloma is characterized by excessive numbers of abnormal plasma cells in the bone marrow and overproduction of intact monoclonal immunoglobulin (IgG, IgA, IgD, or IgE) or Bence-Jones protein (free monoclonal K and A light chains). Hypercalcemia, anemia, renal damage, increased susceptibility to bacterial infection, and impaired production of normal immunoglobulin are common clinical manifestations of multiple myeloma. It is often also characterized by diffuse osteoporosis, usually in the pelvis, spine, ribs and skull.

According to one specific embodiment, treatment or prevention of a proliferative disorder, specifically a non-solid tumor such as multiple myeloma, may be reflected in reduction in tumor load (quantified by determination of serum paraprotein), plasmacytosis, and bone lesions. For example, treatment with MEDICA drugs may reduce tumor load, plasmacytosis, and bone lesions by at least 5%, at least 10%, at least 15%, at least 20%, at least 25%, at least 30%', at least 40%, at least 50% or even by at least 55 or 60% as compared to these parameters prior to treatment.

According to a second aspect, the invention relates to a method of treatment or prevention of an immune-related disorder. The method of the invention comprises the step of administering to a subject in need thereof a therapeutically effective amount of at least one long-chain substituted amphipathic carboxylate or any salt, ester or amid thereof or any combination or mixture thereof, or of any composition comprising the same. It should be noted that such composition may optionally further comprises at least one pharmaceutically acceptable carrier, diluent, excipients and/or additive.

According to one embodiment, the method of the invention may optionally further comprises administration of at least one additional therapeutic agent. Still further, such therapeutic agent may be comprised within the same composition with the MEDICA drugs.

According to one specific embodiment, the method of the invention may use any of the long-chain optionally substituted amphipathic dicarboxylic acid descried by the invention. More specifically, these long-chain substituted amphipathic carboxylate may be any one of the 3,3,14,14-tetramethyl-hexadecanedioic acid (M16ββ), the 2,2,15,15-tetramethyl-hexadecanedioic acid (M16αα) and the 4,4,15,15-tetramethyl-octadecanedioic acid (M18γγ) representatives.

According to one embodiment, the invention provides a method for the treatment or prevention of an immune-related disorder such as an inflammatory disease, an autoimmune disease and malignant and non-malignant proliferative disorders.

The present inventors have previously hypothesized that MEDICA drugs act as inhibitors of the HNF-4α transcription factor and therefore may be used for the treatment of HNF-4α-mediated disorders. Therefore, according to a specifically preferred embodiment, the invention relates to the use of MEDICA drugs for treating immune-related disorders that are not mediated, associated with, caused by, linked to, usually occurring together and believed to have an impact on HNF-4α.

According to one embodiment, the inflammatory disease treated by the method of the invention may be any one of multiple sclerosis, an atherosclerotic disease, an inflammatory bowel diseases and arthritis.

In yet another embodiment, the method of the invention is intended for the treatment and prevention of an atherosclerotic disease. Such disease may be cardiovascular disease, cerebrovascular disease or peripheral vessel disease.

According to another embodiment, the method of the invention is specifically applicable for the prevention and the treatment of multiple sclerosis.

In another embodiment, the method of the invention is used for preventing and treating IBD, for example, colitis or Crohn's disease.

According to another specific embodiment, the method of the invention is applicable for the prevention and the treatment of a malignant disorder. A malignant proliferative disorder may be any one of solid and non-solid tumor selected from the group consisting of carcinoma, sarcoma, melanoma, leukemia and lymphoma. More particularly, the malignant disorder may be hepaotcellular carcinoma, melanoma, colon cancer, myeloma, acute or chronic leukemia.

As used herein to describe the present invention, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods and compositions of the present invention may be used in the treatment of non-solid and solid tumors.

According to one specific embodiment, the method of the invention may be used for treatment of colon cancer.

In yet another specific embodiment, the method of the invention may be used for the treatment and prevention of Multiple myeloma.

Another embodiment of the invention relates to the use of the method of the invention for treating or preventing autoimmune disease, for example, rheumatoid arthritis, diabetes, asthma, acute and chronic graft versus host disease, systemic lupus erythmatosus, scleroderma and immune mediated hepatitis.

“Treatment” refers to therapeutic treatment. Those in need of treatment are mammalian subjects suffering from any pathologic condition involving immune system abnormalities. By “patient” or “subject in need” it is meant any mammal who may be affected by the above-mentioned conditions, and to whom the treatment and diagnosis methods herein described is desired, including human, bovine, equine, canine, murine and feline subjects. Preferably said patient is a human. Administering of the drug combination to the patient includes both self-administration and administration to the patient by another person.

According to another specific embodiment, the active ingredients used by the invention or any composition thereof, may be administered via any mode of administration. For example, oral, intravenous, intramuscular, subcutaneous, intraperitoneal, parenteral, transdermal, intravaginal, intranasal, mucosal, sublingual, topical, rectal or subcutaneous administration, or any combination thereof.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician.

In yet another embodiment, a preferred therapeutically effective amount of MEDICA drugs administered daily by the method of the invention, may range from about 0.05 mg/kg to about 10 mg/kg of body weight, specifically, between about 0.10 to 8, 0.20 to 6, 0.30 to 5 mg/kg. According to a specific embodiment, the effective amount may be any one of 5, 10, 20, 30, 40, 50, 60, 70, 80, 90, 100, 150, 200, 250, 300, 350, 400, 450 and 500 mg, preferably, per day. Specifically, the effective amount may be about 10 to 400 mg per day, more specifically, any one of 10, 60, 150 and 400 mg per day. It should be appreciated that such effective amount is specific for a human subject. Still further, it should be recognized that these “human doses” are calculated by dividing doses (mg/kg) in mice by about 12 to derive respective Human Equivalent Doses (HED) (mg/kg), and further divided by 10 (Safety Factor in extrapolating from mice to human), in accordance with the Guidance for Industry, Estimating the Maximum Safe Starting Dose in Initial Clinical Trials for Therapeutics in Adult Healthy Volunteers. [Food and Drug Administration (FDA) and Center for Drug Evaluation and Research (CDER), July (2005)]. This effective amount of MEDICA drug is preferably comprised within a dosage unit form. Additionally, the administration of the MEDICA drugs according to the invention may be periodically, for example, the periodic administration may be effective twice daily, three times daily or at least one daily for at least about three days to three months. The advantages of lower doses are evident to those of skill in the art. These include, inter alia, a lower risk of side effects, especially in long-term use, and a lower risk of the patients becoming desensitized to the treatment.

According to another embodiment, the dosage unit form used by the method of the invention may be either for a single or for repeated administration. According to another embodiment, administration of said dosage unit form is repeated every one to five, ten or twenty four hours, for a therapeutically sufficient period of time. According to an alternative embodiment, the dosage unit form may be a sustained-released dosage unit form which provides continues pH independent drug release for a considerable period of time after administration.

It should be noted that while treatment of other adverse indications may be effected using doses of MEDICA drugs in the range of from about 10 mg per day to about 500 mg per day and/or may be effected following at least between one days to about treatment for life. In another embodiment, treatment using the MEDICA drugs may be effected following at least 1, 2, 3, 4, 5, 6, 7, 8, 9, 10, 14, 30, 60, 90 days of treatment, and proceeding on to treatment for life.

It should be noted that the treatment of different conditions may indicate the use of different doses or different time periods; these will be evident to the skilled medical practitioner.

For example, for treating MS, a specific dose may be between about 0.1 to 10 mg/kg/day, specifically, 1.0 mg/kg/day. The periodic administration may be effected twice daily, three times daily, or at least twice weekly for at least about one day to six months, specifically for at least ninety days. Treatment may start at least about three days before occurrence of disease episode symptoms.

In yet another embodiment, for treating colitis or Crohn's disease, a specific dose may be between about 0.1 to 10 mg/kg/day, specifically, 6.0 mg/kg/day for about one to ten days and 3.0 mg/kg/day for about one to thirty days.

Another embodiment relates to the treatment of atherosclerosis, a specific dose may be between about 0.1 to 10 mg/kg/day, specifically, 1.0 mg/kg/day. The periodic administration may be effected twice daily, three times daily, or at least twice weekly for at least about six months to six years.

Another embodiment relates to the treatment of multiple myeloma, a specific dose may be between about 0.1 to 10 mg/kg/day, specifically, 1.0 mg/kg/day. The periodic administration may be effected twice daily, three time daily, or at least one daily for at least about one day to three months or throughout remission periods.

A further embodiment relates to the treatment of colon cancer, a specific dose may be between about 0.1 to 10 mg/kg/day, specifically, 1.0 mg/kg/day. The periodic administration may be effected twice daily, three time daily, or at least one daily for at least about one day to three months or throughout remission periods.

As indicated above, therapeutic formulations may be administered in any conventional dosage formulation. Formulations typically comprise at least one active ingredient, as defined above, together with one or more acceptable carriers thereof.

The MEDICA drugs or any composition of the invention may be preferably administered orally. The active combined drug compounds employed in the instant therapy can be administered in various oral forms including, but not limited to, tablets, capsules, pills, powders, granules, elixirs, tinctures, suspensions, syrups, and emulsions. It is contemplated that the active drug compounds can be delivered by any pharmaceutically acceptable route and in any pharmaceutically acceptable dosage form. These include, but are not limited to the use of oral conventional rapid-release, time controlled-release, and delayed-release pharmaceutical dosage forms. The active drug components can be administered in a mixture with suitable pharmaceutical diluents, excipients or carriers (collectively referred to herein as “carrier” materials) suitably selected to with respect to the intended form of administration. As indicated, it is contemplated that oral administration can be effectively employed. Thus, tablets, capsules, syrups, and the like as well as other modalities consistent with conventional pharmaceutical practices can be employed.

In instances in which oral administration is in the form of a tablet or capsule, the active drug components can be combined with a non-toxic pharmaceutically acceptable inert carrier such as lactose, starch, sucrose, glucose, modified sugars, modified starches, methylcellulose and its derivatives, dicalcium phosphate, calcium sulfate, mannitol, sorbitol, and other reducing and non-reducing sugars, magnesium stearate, stearic acid, sodium stearyl fumarate, glyceryl behenate, calcium stearate and the like. For oral administration in liquid form, the active drug components can be combined with non-toxic pharmaceutically acceptable inert carriers such as, ethanol, glycerol, water and the like. When desired or required, suitable binders, lubricants, disintegrating agents and coloring and flavoring agents can also be incorporated into the mixture. Stabilizing agents such as antioxidants, propyl gallate, sodium ascorbate, citric acid, calcium metabisulphite, hydroquinone, and 7-hydroxycoumarin can also be added to stabilize the dosage forms. Other suitable compounds can include gelatin, sweeteners, natural and synthetic gums such as acacia, tragacanth, or alginates, carboxymethylcellulose, polyethylene, glycol, waxes and the like.

Alternatively, the MEDICA drugs used by the invention may also be administered in controlled release formulations such as a slow release or a fast release formulation. Such controlled release formulations may be prepared using methods well known to those skilled in the art. The method of administration will be determined by the attendant physician or other person skilled in the art after an evaluation of the subject's conditions and requirements.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art Methods of preparing various pharmaceutical compositions with a certain amount of active ingredient are known, or will be apparent in light of this disclosure, to those skilled in this art.

The preventive effect of the MEDICA drugs has been demonstrated by Example 5, particularly for multiple sclerosis, using the EAE model. Therefore, the invention further provides a method for preventing or reducing the risk of developing an immune-related disorder. Such method comprises the administration of a prophylactically effective amount of at least one long-chain optionally substituted amphipathic carboxylate or any salt, ester or amid thereof or any combination or mixture thereof or of any composition comprising the same, to a person at risk of developing an immune-related disease.

The term “prophylactically effective amount” is intended to mean that amount of a the MEDICA drugs of the invention or a pharmaceutical composition comprising the same, that will prevent or reduce the risk of occurrence or recurrence of the biological or medical event that is sought to be prevented in a tissue, a system, animal or human by a researcher, veterinarian, medical doctor or other clinician.

It should be noted that for the method of treatment and prevention provided in the present invention, said therapeutic effective amount, or dosage, is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. In general, dosage is calculated according to body weight, and may be given once or more daily, weekly, monthly or yearly, or even once every 2 to 20 years. Persons of ordinary skill in the art can easily estimate repetition rates for dosing based on measured residence times and concentrations of the MEDICA drug used by the invention or any composition of the invention in bodily fluids or tissues. Following successful treatment, it may be desirable to have the patient undergo maintenance therapy to prevent the recurrence of the disease state, wherein the combined composition of the invention is administered in maintenance doses, once or more daily.

According to a third aspect, the invention relates to the use of a therapeutically effective amount of at least one long-chain substituted amphipathic carboxylate or any salt, ester or amid thereof or any combination or mixture thereof, in the preparation of a medicament for the treatment or prevention of an immune-related disorder.

In yet another embodiment, such immune-related disorder may be any one of an inflammatory disease, an autoimmune disease and malignant and non-malignant proliferative disorders.

According to another embodiment, the present invention further provides an oral pharmaceutical composition comprising a therapeutically effective amount of at least one long-chain substituted amphipathic carboxylate and optionally at least one additional therapeutic agent for the concerned indication, with a pharmaceutically acceptable carrier. It should be noted that where the MEDICA drugs and optionally the additional therapeutic agent are formulated in an enteric coated dosage form, a substantial release of the compound from the dosage form after oral administration to a patient is delayed until passage of the dosage from through the stomach.

Moreover, different combinations of different ratios at different concentrations of at least one long-chain substituted amphipathic carboxylate (MEDICA drugs) may be used for different disorders. A daily dose of the active ingredients in a preferred mixture may contain between about 10 to 500, preferably, 30 to 100 mg per day of MEDICA drug/s and optionally an additional therapeutic agent. It should be appreciated that any quantitative ratio may be used. For example: 1:1000, 1:2, 1:50, 1:200, 1:350, 1:500 and any possible combination.

Therefore, disclosed herein is a therapeutic composition that contains at least one therapeutically active form of a long-chain substituted amphipathic carboxylate (also referred to as MEDICA drug) and optionally at least one additional other therapeutic agent.

The additional therapeutic agent may be capable of addressing at least one immune-related abnormality or disorder.

Still further, the present invention particularly relates to additive and synergistic combinations of MEDICA drugs and additional therapeutic agent, or of pharmaceutically acceptable salts thereof, whereby those additive and synergistic combinations are useful in preventing or treating subjects suffering from an immune-related disorder, specifically, an inflammatory disorder, atherosclerotic disease, autoimmune disorder or a malignant and non-malignant proliferative disorder. The synergistic and additive compositions of the invention may also be used for the prevention or the treatment of subjects presenting with symptoms or signs of such disorders.

By synergic combination is meant that the effect of both MEDICA drugs and the additional therapeutic agent is greater than the sum of the therapeutic effects of administration of any of these compounds separately, as a sole treatment. Alternatively, synergic combination may allow for an additive therapeutic effect in the absence of side effects due drug-drug interaction.

The compositions of the invention generally comprise a buffering agent, an agent which adjusts the osmolarity thereof, and optionally, one or more pharmaceutically acceptable carriers, excipients and/or additives as known in the art. Supplementary active ingredient can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

The MEDICA drugs of the present invention, optionally combined with an additional therapeutic agent, may generally be administered in the form of a pharmaceutical composition together with a pharmaceutically acceptable carrier or diluent. Thus, both compounds used (MEDICA drugs and optionally an additional therapeutic agent) by this invention can be administered either individually in a kit or together in any conventional oral, parenteral or transdermal dosage form.

More particularly, since the present invention relates to the treatment of diseases and conditions with the active ingredients which may be administered separately, the invention therefore further provides combining separate pharmaceutical compositions in kit form. The kit includes two separate pharmaceutical compositions: long-chain substituted amphipathic carboxylate (MEDICA drugs) and an additional therapeutic agent. The kit includes container means for containing both separate compositions; such as a divided bottle or a divided foil packet however, the separate compositions may also be contained within a single, undivided container. Typically the kit includes directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

Still further, the invention provides a method of treatment of an immune-related disorder comprising the step of administering to a subject in need thereof a therapeutically effective amount of a first and a second unit dosage forms comprised in the kit according to the invention.

It should be appreciated that both components of the kit, the MEDICA drugs in the first dosage form and the additional therapeutic agent in the second dosage form may be administered simultaneously.

Alternatively, said first compound or dosage form and said second compound or dosage form are administered sequentially in either order.

The invention further provides a method for preventing or reducing the risk of developing an immune-related disease comprising the administration of a prophylactically effective amount of a first and a second unit dosage forms comprised in the kit of the invention, to a person at risk of developing atherosclerotic disease.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention

### EXAMPLES

### Experimental Procedures

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Md. (1988).

Standard organic synthesis protocols known in the art not specifically described herein are generally followed essentially as in Organic syntheses: Vol. 1-79, editors vary, J. Wiley, New York, (1941-2003); Gewert et al., Organic synthesis workbook, Wiley-VCH, Weinheim (2000); Smith & March, Advanced Organic Chemistry, Wiley-Interscience; 5th edition (2001). Standard medicinal chemistry methods known in the art not specifically described herein are generally followed essentially as in the series “Comprehensive Medicinal Chemistry”, by various authors and editors, published by Pergamon Press.

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Vanderkerken K The 5T2MM murine model of multiple myeloma: maintenance and analysis. [Methods Mol. Med. 113:191-205 (2005); Epstein J. The SCID-hu myeloma model. Methods Mol. Med. 113:183-90 (2005)].

### Tested MEDICA Drugs

M16αα, (M2001)-2,2,15,15-tetramethyl-hexadecanedioic acid, molecular weight—342, batch No. —04/00100, purity—99.8%;
M16ββ (M1001)—molecular weight—386, batch No. —04/00200, purity—99.3%), both were supplied by the SyndromeX Ltd. (Jerusalem; Isarel).

### Cell Lines

*HT-29 colon cancer cells (ATCC number HTB-38).

Cells were cultured in Dulbecco's Modified Eagle Medium (DMEM) supplemented with 10% fetal calf serum, glutamine and antibiotics (100,000 units/L penicillin and 100 mg/L streptomycin sulfate).

*The U-266 and RPMI-8226 cell lines serve as cell models for MM [Reviewed in Kovacs E, The Scientific World Journal 6:888-898 (2006)].

*The 5T33 or 5T2mM murine myeloma cells are provided by Dr. J. Radl (TNO Institute Leiden, The Netherlands). The cells are passaged at 0.5×10⁶ cells/mL concentration in 20 mL minimal essential medium (MEM) containing 2 mM L-glutamine, 100 mM nonessential amino acids, 100 mM sodium pyruvate (all from GIBCO-Invitrogen, Invitrogen AB, Stockholm, Sweden), 5×10⁻⁵ M 2-mercaptoethanol, and 10% heat-inactivated fetal calf serum (FCS). The cells are incubated at 37° C., 5% CO₂, 95% humidity and the medium is changed every 2 to 4 days in 75-cm² flasks. Aliquots of early passaged cells are frozen in 10% dimethylsulfoxide, 90% FCS and stored at −140° C. for later reconstitution. Morphology of the cells in culture is observed using an inverted microscope with a UV module (Olympus) at every passage.

### Animal Models

### A. Colon Carcinoma

Balb-c/nude mice are injected with HT-29 cells in the right thigh and are treated with either powdered regular chow, 0.5% (W/W) M-16ββ in chow, or 6.5% (W/W) M16αα in chow or 0.5% (W/W) M18γγ in chow.

Tumours are measured by calliper every other day. Mice are sacrificed three weeks after being inoculated with HT-29 cells. The tumours are excised, weighed, sampled in lysis buffer for antigen profiling (bak, 14.2, caspase 3, cytochrome c, PCNA) and the remaining was fixed in buffered formalin for histopathology.

### B. Experimental Colitis

Eight weeks old Balb/C male mice weighing 25 gr (Harlan Inc. Jerusalem, Israel) were housed under normal conditions with free access to water and rodent laboratory chow.

Colitis was induced by 5% DSS (Dextran Sodium Sulfate, m.w 36,000-50,000) added to their drinking water for 7 days. M16αα dissolved in 1% CMC (Carboxy Methyl Cellulose) was administered daily by gavage, starting 2 days prior to DSS and through the 7 days of DSS administration. Dosing: 60 mg/kg/day, for 5 days followed by 30 mg/Kg/day for the last 4 days. Control mice were gavaged with the 1% CMC vehicle.

Upon sacrifice, the entire colon was resected, weighed and its length measured.

### The Disease Activity Index (DAI)

DAI was defined by the appearance of Diarrhea, Internal bleeding and External bleeding, as indicated in Experimental procedures. 0=none; 1=mild; 2=gross.

### Colitis Pathological Score

Extent of colitis (None(0), Focal(1), Segmental(2), Multisegmental(3)), Inflammation grade (0-3), Damage/Necrosis (None(0), Superficial(1), Muscularis mucosa(2), Muscularis propria(3)) and Re-epithelializaion. (None(3), Focal(2), Mutifocal(1), Complete re-epithelializtion (0)).

Frozen sections of the intestine were homogenized by Polytron in lysis buffer (50 mM Tris-Hcl Ph 8.0 containing 0.5% NP-40, 1 mM EDTA, 150 mM NaCL, 10% glycerol, 1 mM PMSF and protease inhibitor cocktail) and were analyzed by Western blot

### C. EAE Model for MS

EAE was induced in 8-week-old female C57BL/6 mice by injecting s.c. into the left para-lumbar region 125 μg of myelin oligodendrocyte glycoprotein 35-55 peptide (MOG35-55) emulsified in complete Freund's adjuvant (CFA) containing 5 mg/ml heat-killed Mycobacterium tuberculosis. Immediately thereafter, and, again, at 48 h, the mice were inoculated i.p. with 0.5 ml of pertussis toxin (400 ng). 7 days later the mice were further challenged with an additional injection of MOG35-55 peptide in CFA injected into the right para-lumbar region. Mice were treated with M16αα or vehicle (1% CMC) as indicated.

### Evaluation of EAE Clinical Score

0—without clinical disease;
1—tail weakness;
2—hind limb weakness sufficient to impair righting;
3—one limb plagic;
4—paraplegia with forelimb weakness;
5—quadriplegia;
6—death.

### D. Model for MM.

Eight to ten weeks old female and male C57BL/KaLwRij, mice are supplied by Harlan (The Netherlands) and housed under conventional conditions including access to tap water and standard chow ad libitum. Groups of C57BL/KaLwRij mice (10-15 mice/group are injected i.v. with 10⁴ and/or 10⁵ 5T33 mM cells suspended in a total volume of 100 μL sterile PBS/mouse. Control mice (4-6 mice/group) are either left un-injected or injected with equal volume of PBS i.v. From day 5 after cell injection, the animals are examined twice daily for the development of paraplegia. At weekly intervals (in the kinetics study) and at the time of disease development, the mice are killed by CO₂ inhalation and the spleens, livers, thymi, and lymph nodes are excised and kept in PBS until processing for preparation of single-cell suspensions. Bone marrow from femora and tibiae are obtained by flushing PBS into the cavity of bones. Tumor load is quantified by determination of serum paraprotein by ELISA, or by capillary zone electrophoresis (Paragon). Plasmacytosis is determined by FACS analysis. Bone lesions are determined by histmorphometry (H&E).

Cell proliferation is measured by cell proliferation reagent WST-1 (Roche Diagnostics, Mannheim), 5T33 mM cells at the cell concentrations of 1×10⁴ and/or 2×10⁴ cells/well are seeded into a 96-well tissue culture plate in a final volume of 100 μL of cell culture medium. 5×10⁻⁵ M 2-mercaptoethanol, and 10% heat-inactivated fetal calf serum (FCS). Thereafter, 10 μL of the cell proliferation reagent WST-1 is added to each well and the cells were incubated for 4 hours at 37° C. and 5% 002. Then, the absorbance of the samples against a blank control is measured using a microplate spectrophotometer ELISA reader with 420 nm as detection wavelength and 650 nm as reference wavelength for WST-1 assay. Triton-X treated cells are used as reference and positive controls, respectively. Cell survival is calculated as the percent ratio of samples' absorbance against controls.

### TUNEL Assay

Samples fixated in formalin are embedded in paraffin, sectioned (5 μm thick) and analyzed for terminal deoxynucleotidyl transferase-mediated dUTP-nick end labeling (TUNEL) (Klenow-Frag-El, Oncogene, Cambridge, Mass.), according to the manufacturers instructions.

### Example 1

### The Anti-Inflammatory Effect of MEDICA Drugs

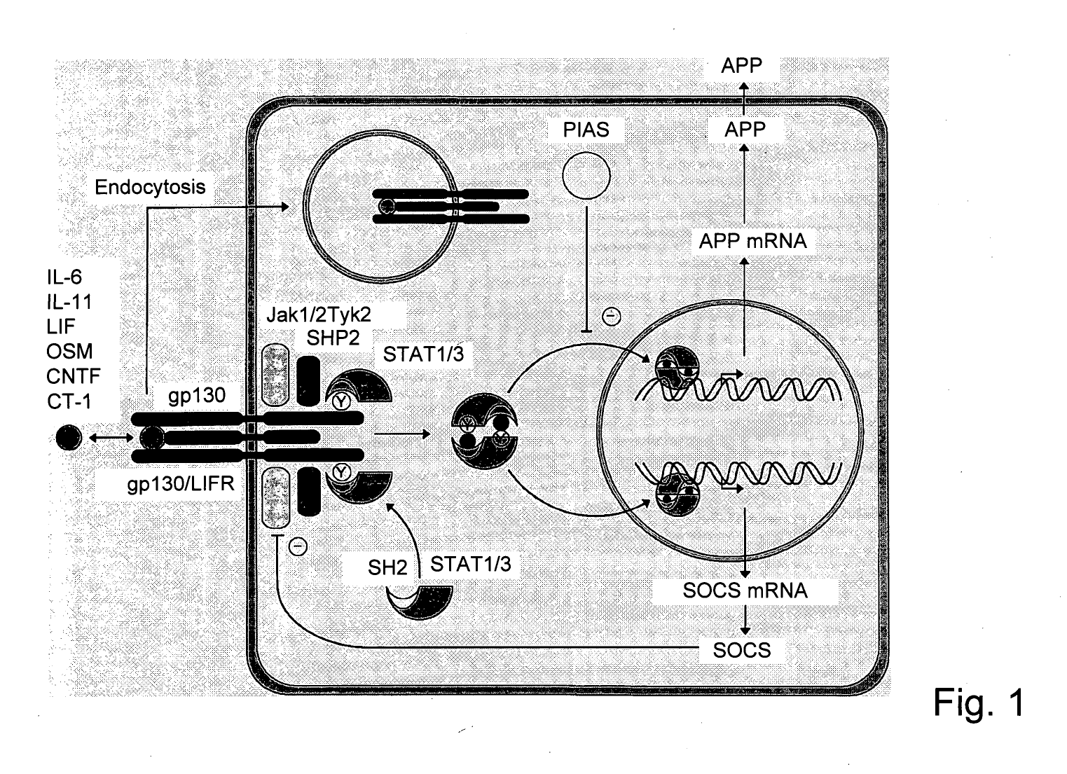

Acute phase proteins (APP) (e.g., CRP, Serum amyloid A1 (SAA1), fibrinogen), participate in inflammatory signaling and derive mainly from hepatocytes in response to interleukin-6 (IL-6) and other cytokines. APP are then secreted into the systemic circulation. Recent clinical trials showed that CRP, SAM and fibrinogen are powerful independent predictors of future cardiovascular events. As illustrated by FIG. 1, the signaling pathway that leads to IL-6-induced CRP expression in hepatocytes requires the binding of IL-6 to its cognate receptors. Binding of IL-6 to its cell surface receptor, IL-6Rα, induces the formation of a complex with the signal-transducing molecule, gp130. The IL-6Rα/gp130 complex further induces the activation and phosphorylation of the JAK kinases, leading to downstream phosphorylation of STAT3, its nuclear translocation and induction of the expression of the acute phase reactants.

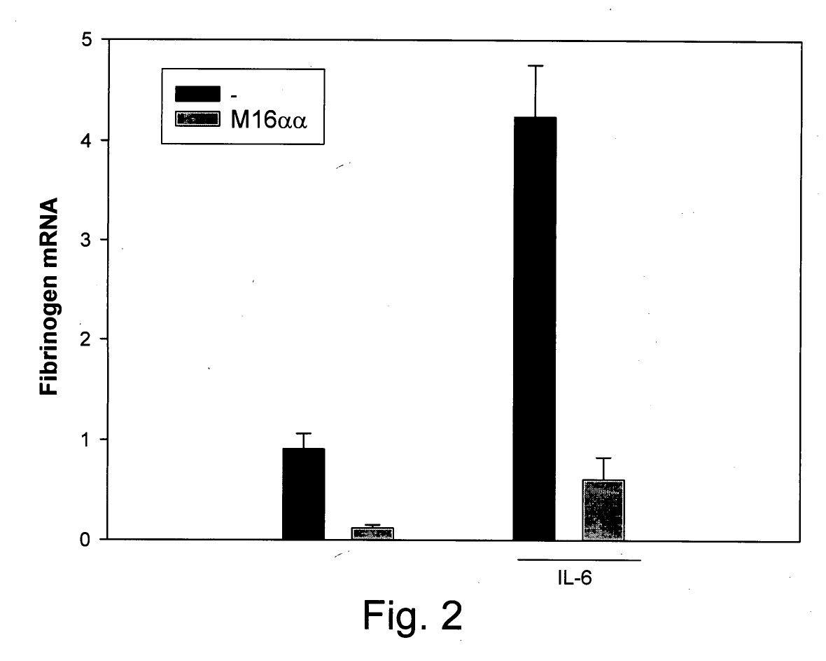

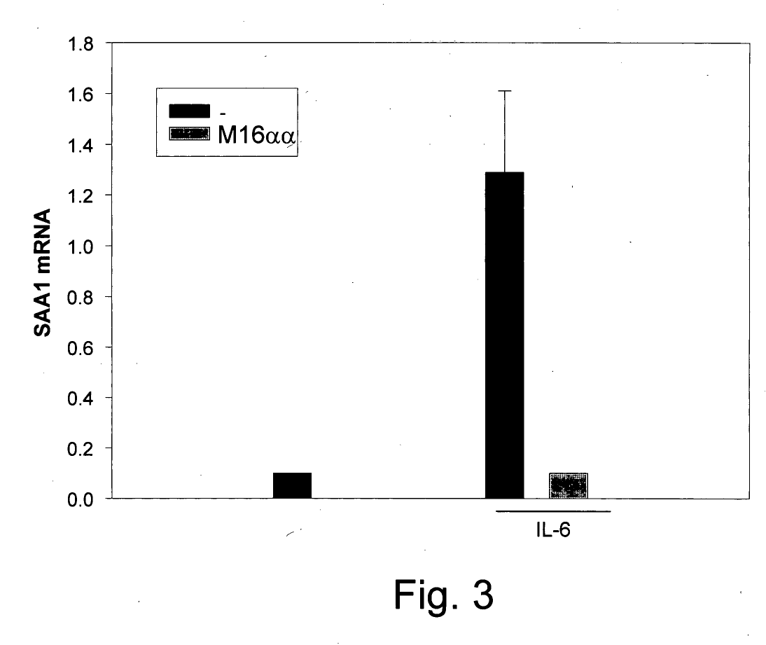

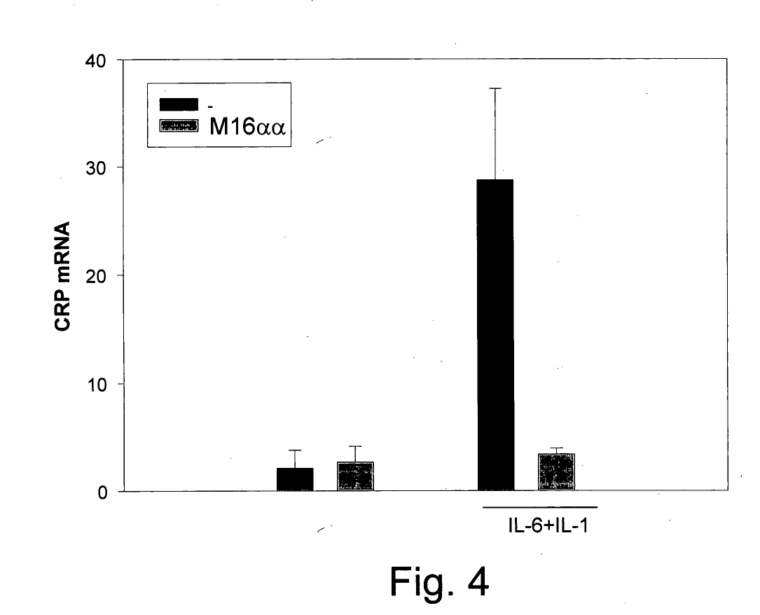

The hypolipidemic effect of both M16ααα and/or M16ββ (MEDICA drugs) and its application in the treatment of Metabolic Syndrome, led the inventors to try and examine the effect of these drugs on another crucial risk factor in Metabolic Syndrome CVD patients, namely, the inflammatory indications. These indications are identified by markers like C-reactive protein (CRP), Serum amyloid A (SAA), fibrinogen and ICAM that promote atherosclerotic CVD. To evaluate the possible anti-inflammatory effect of MEDICA drugs, the inventors first examined the effect of M16αα, on the expression of different acute phase proteins (APP). Human liver cells (HepG2 cells) were stimulated with IL-6 and incubated with the MEDICA drug, M16αα. Total RNA was analyzed by real time PCR for expression of mRNA of different acute phase proteins (APP). As demonstrated by FIG. 2, IL-6 leads to increase in fibrinogen mRNA levels. Treatment with 200 mM M16αα, clearly reduced fibrinogen mRNA levels, in control as well as IL-6 induced cells. Similar reducing effect of M16αα was also demonstrated for SAA1 and CRP mRNA prepared from IL-6 and IL-1+IL-6 induced cells, respectively (FIGS. 3 and 4). These results clearly indicate that M16αα decreases basal as well as cytokine-induced APP expression.

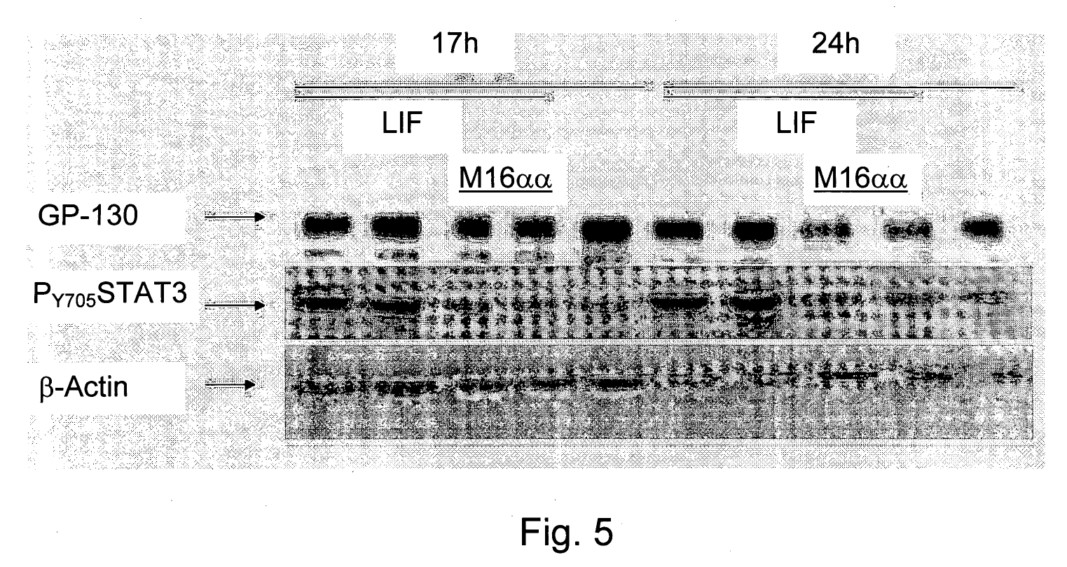

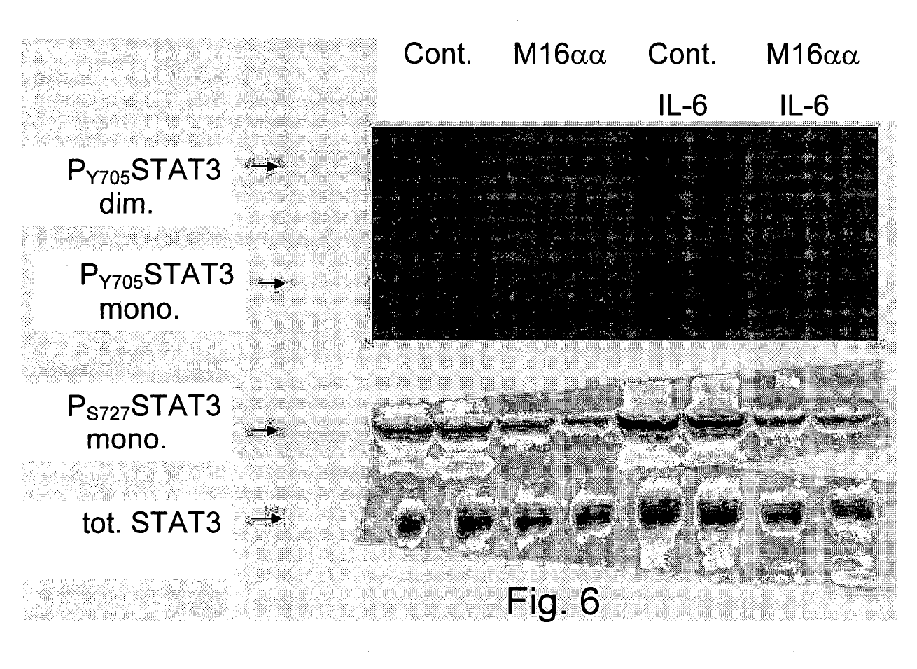

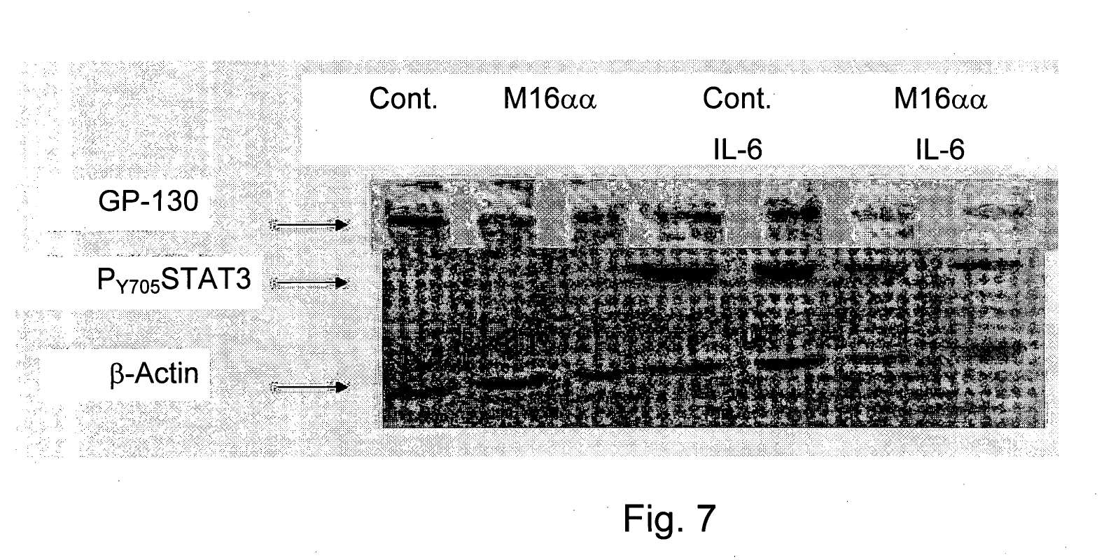

The examined effect of MEDICA drugs on expression of APP, was further analyzed by analyzing the effect of M16αα on downstream elements in the signaling pathway leading to expression of APP. Signal Transducers and Activators of Transcription 3 (STAT3), is a transcription factor phosphorylated by JAK kinases in response to cytokine activation of a cell surface receptor tyrosine kinases. Cytokines that activate STAT3 include growth hormone, IL-6 family cytokines, Oncostatin, LIF, CNTF and G-CSF, Leptin and others. As illustrated by FIG. 1, upon cytokine activation, the STAT3 proteins dimerize and are localized to the nucleus where they activate transcription of APP as well as of other cytokine-responsive genes coding for cell survival, growth and proliferation. Therefore, the inventors next examined whether the effect of MEDICA drugs on the expression of APP is a result of interfering with STAT3 transcription activity or whether MEDICA drugs are involved in a more upstream element of the transduction pathway such as the gp130 receptor. Since dimerization and activation of STAT3 is dependent on its phosphorylation in both, tyrosine 705 (pY⁷⁰⁵) and serine 727 (pS⁷²⁷) residues, the inventors next examined the effect of M16αα on the phosphorylation of STAT3. Thus, HepG2 cells were incubated with M16αα (200 μM) for 17 and 24 h. At each time point LIF (20 ng/ml) was added for the last 20 minutes or for the last 3 h. Western blots of cell lysates were reacted with anti gp130 and anti P-STAT3 (Y705) antibodies and normalized by β-actin. As shown by FIG. 5, treatment with M16αα, clearly inhibits the LIF-induced phosphorylation of tyrosine 705 residue of STAT3. As shown by this figure, M16αα also significantly reduced gp130 protein levels. Similar results were also demonstrated for IL-6-induced phosphorylation of STAT3 tyrosine 705 residue, as shown by FIG. 6. Examination of the effect of M16αα on phosphorylation of STAT3 serine 727 residue; reveled that also the phosphorylation of 5727 was significantly inhibited by M16αα (FIG. 6). It should be noted that expression of STAT3 as shown by Western blot of the total STAT3 was not affected by M16αα, which only reduces the phosphorylation of STAT3 in both, Y705 and 5727, but not the expression of said protein (FIG. 6). Inhibition of IL-6-induced phosphorylation of STAT3 Y705, was also demonstrated using kidney cells (Hek293), as shown by FIG. 7.

The inhibitory effect of M16αα on the phosphorylation, but not the expression of STAT3, indicates that M16αα may affect a molecule which is upstream to STAT3 in this pathway. As shown by FIGS. 5-7, treatment of M16αα results in reduction in the protein levels of gp130, which is the tyrosine kinase receptor upstream to STAT3 in this pathway. The inventors thus next examined whether M16αα affects gp130 at the transcription level. Therefore, HepG2 cells were incubated with M16αα (200 μM) for up to 40 h. Total RNA was prepared at 2 h, 4 h, 8 h, 24 h and 40 h, and gp130 transcription was analyzed by real time RT-PCR and normalized with GAPDH. The results clearly indicated that gp130 transcript levels remained unaffected by M16αα.

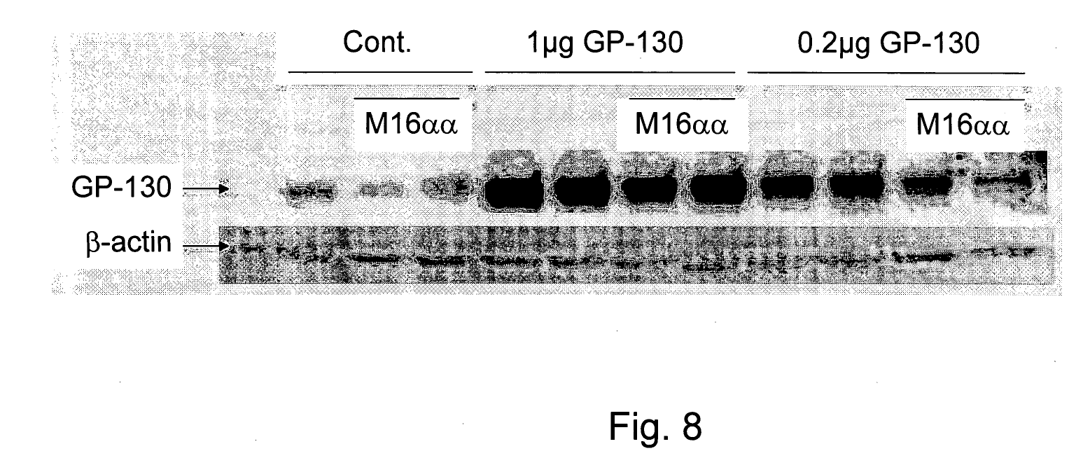

In order to examine the mode of action of M16αα in reducing gp130 protein levels, Hek293 cells were transfected with an expression vector for the gp130 protein. Following 20 h after transfection, M16αα (200 mM) was added for 9 h. Western blots of cells lysates were reacted with anti-gp130 antibodies and normalized by β-actin. As shown by FIG. 8, treatment with M16αα resulted in clear reduction of gp130 protein levels. This effect was clearer when smaller amounts of plasmid were used for transfection (0.2 microgram).

Therefore, M16αα-induced decrease of overexpressed gp130 specifically indicate that MEDICA drugs may either induce the degradation of gp130 or suppress gp130 translation, and as a result, the phosphorylation of STAT3 (Y705, 5727) is inhibited. Inhibition of STAT3 phosphorylation inhibits its transport into the nucleus, resulting in suppression of the expression of STAT3-responsive genes (e.g., APP).

As shown above, MEDICA drugs reduce levels of APP, by affecting the protein levels of the gp130 receptor. These findings furnish new evidence for direct anti-inflammatory properties of MEDICA drugs and provide new insight into their clinical benefits, alone or in combination with other immuno-modulatory agents.

### Example 2

### Suppression of Colon Cancer by MEDICA Drugs

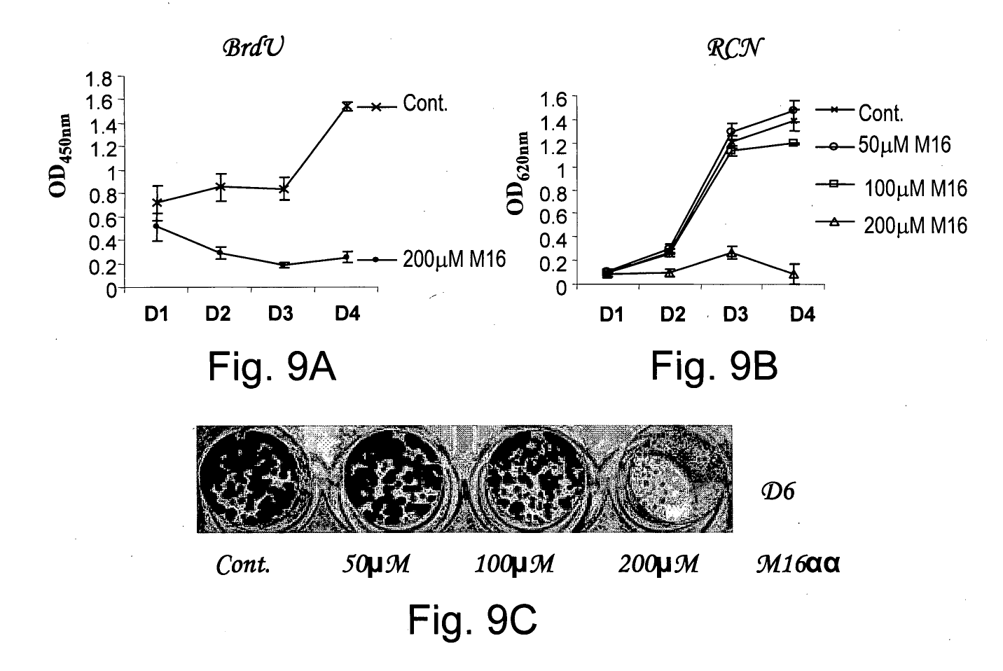

The involvement of MEDICA drugs in signal transduction pathways related to immuno-modulatory mechanisms have led the inventors to further investigate the effect of the MEDICA drugs in different disorders associated with abnormal immune reaction. Since cancerous processes may be promoted as a result of interruption of the Th1/Th2 balance, the possible beneficial effect of the MEDICA drugs on tumor proliferation and progression have been next examined. The effect of different concentrations M16αα on cell proliferation was first examined in vitro using the HT29 colon cancer cells as a model for colon cancer. Cells were cultured in the presence of different concentrations of M16αα (50, 100, and 200 μM). Samples obtained from days 1, 2, 3 and 6 were analyzed for cell number using the trypan blue exclusion assay. Proliferation was measured by BrdU incorporation assay. As clearly shown by FIG. 9A, treatment of HT29 cells with 200 μM M16αα, significantly reduces BrdU incorporation (FIG. 9A) indicating inhibition of cell proliferation. Analysis of relative cell numbers presented in FIG. 9B, and exemplified by FIG. 9C, clearly indicated significant reduction in cell number of cells treated with 200 μM M16αα. Similar results were also demonstrated with the M16ββ and M18γγ analogs (not shown).

The inhibitory effect of the MEDICA drugs is investigated using an in vivo tumor transplants in Athymic mice, by analyzing tumor growth and induction of apoptosis. Balb-c/nude mice are injected with HT-29 cells in the right thigh and are treated with either powdered regular chow or 0.05% (W/W) M16ββ in chow (about 50 mg/kg body weight/day). Tumours are measured by calliper every other day. Mice are sacrificed three weeks after being inoculated with HT-29 cells, tumors are excised and weighed. For analyzing the effect of M16ββ on induction of apoptosis, tumors are sampled in lysis buffer for antigen profiling (Bak, Bcl-2, caspase-3, cytochrome-c, PCNA, p21, CycD1) and the remaining is fixed in buffered formalin for histopathology and TUNEL assay.

### Example 3

### Suppression of Multiple myeloma (MM) by MEDICA Drugs

Multiple myeloma (MM) is a malignancy of terminally differentiated plasma cells. MM cells localize to the bone marrow, where cell adhesion-mediated autocrine or paracrine activation of various cytokines, such as interleukin 6, insulin-like growth factor 1, and interferon alpha, results in their accumulation mainly because of loss of critical apoptotic controls. Resistance to apoptosis plays a critical role in both pathogenesis and resistance to treatment of MM. Abnormalities in regulation and execution of apoptosis can contribute to tumor initiation, progression, as well as to tumor resistance to various therapeutic agents. Therapeutic modalities that are effective in MM aim at modulating levels of the proapoptotic and antiapoptotic Bcl-2 family of proteins and of inhibitors of apoptosis, expression of which is primarily regulated by STAT3, p53 and nuclear factor kB (NFkB) [Reviewed in Oancea M, et al., Int J Hematol. 80: 224-31 (2004)].

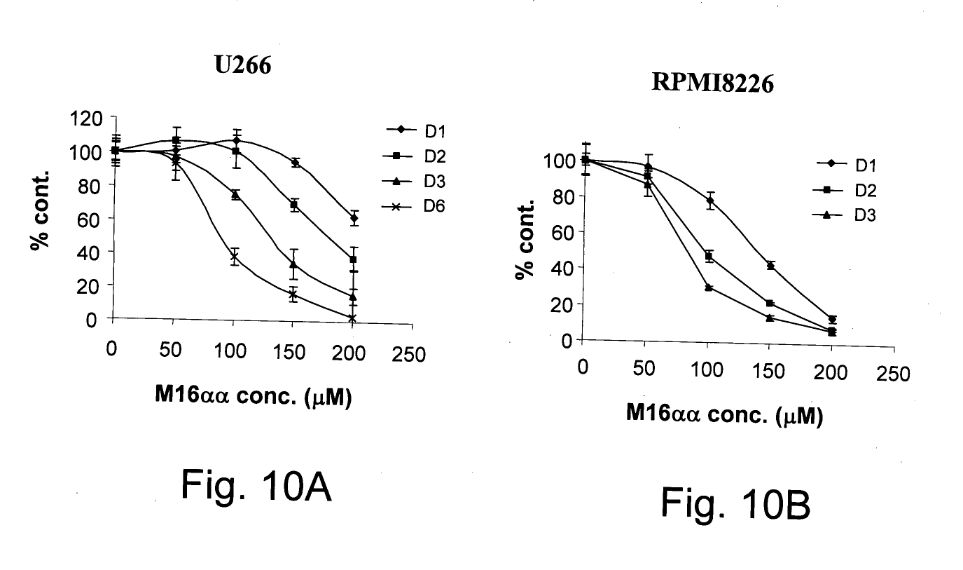

The inhibitory effect of MEDICA drugs on colon tumor cell proliferation, presumably via activation of apoptosis, encouraged the inventors to further investigate the potential beneficial effect of these compounds on other malignancies. The inventors therefore next examined the effect of the MEDICA drugs using the Multiple myeloma model. U-266 and RPMI-8226 cell lines served as cell models for MM. MM cells were incubated in RPMI 1640 medium in the presence of M16αα as indicated. Cell number was determined daily by Trypan Blue exclusion assay. As shown by FIG. 10, in both MM cell lines, the use of M16αα, particularly of 200 μM significantly inhibited cell proliferation. In U266, model (FIG. 10A), complete inhibition of cell proliferation has been demonstrated in day 6, whereas RPMI8226 cells treated with 200 μM M16αα, demonstrated almost a complete inhibition (about 20% of control) has been demonstrated from day one. It should be noted that generally, these cells were more sensitive even to lower concentrations of M16αα.

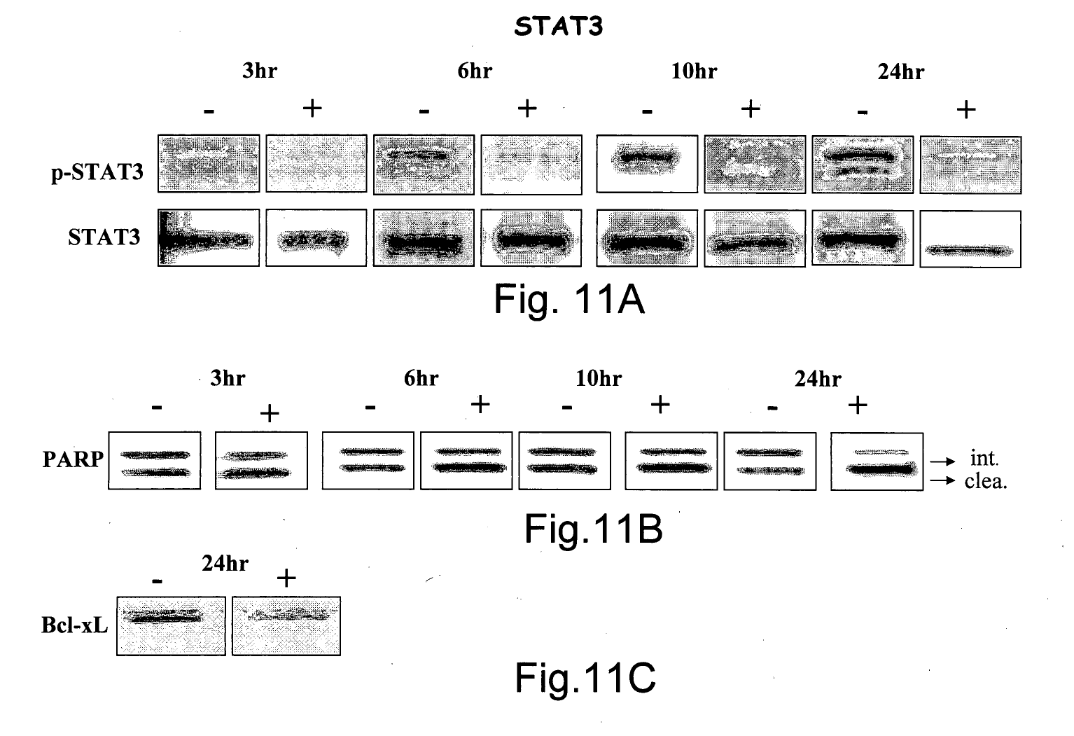

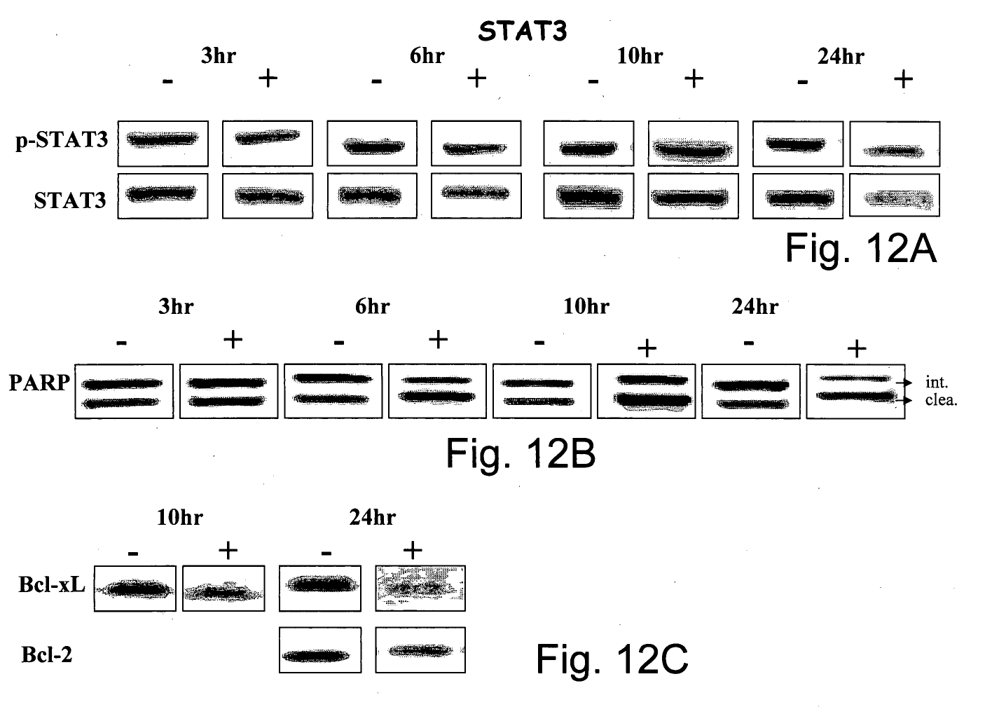

To examine the possibility that the inhibitory effect of M16αα, on Multiple Myeloma may be mediated by enhancement of pro apoptotic mechanism and induction of cell cycle arrest, U266 cells were cultured with 200 μM M16αα in RPMI 1640 medium with 10% FCS for time periods as indicated, lysed, Western blotted and reacted with anti STAT3, p-STAT3 (Y705), PARP or Bcl-xL and Bcl-2 as indicated. As shown by FIG. 11A, treatment with M16αα results in clear reduction of STAT3 phosphorylation, indicating inhibition of the STAT3 pathway by M16αα. FIG. 11B demonstrates enhancement in cleavage of PARP as compared to the intact form expressed in control cells, indicating induction of a pro-apoptotic process by the M16αα, with concomitant decrease in anti-apoptotic markers (Bcl-2, Bcl-xL), demonstrated by FIG. 11C. Similar results for RPM18226 cells cultured with 200 μM M16αα and stimulated with 50 ng/ml IL-6 for 30 min., were demonstrated by FIG. 12.

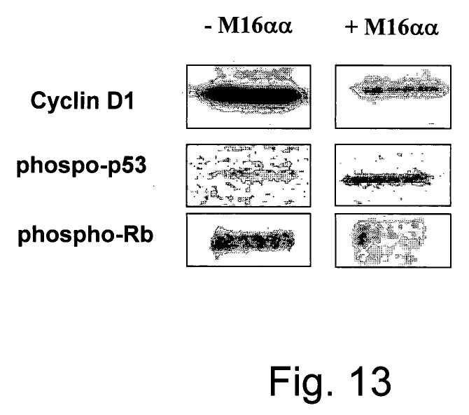

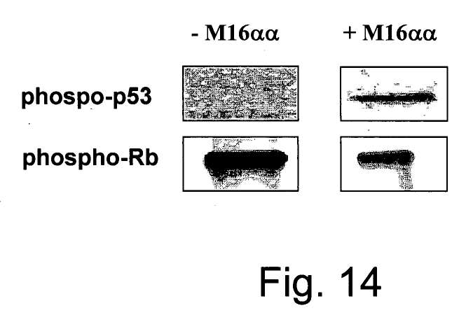

Clear involvement, of induction of cell cycle arrest by the MEDICA drugs is demonstrated for U266 cells treated with 200 μM M16αα, showing reduction in Cyclin D1 expression and in phosphorylated Rb, as well as increase in phosphorylated p53 (FIG. 13). Similar results for RPMI8226 cells showing reduction in phosphorylated Rb, as well as increase in phosphorylated p53 are demonstrated by FIG. 14.

The inhibitory effect of the MEDICA drugs on MM is further investigated in vivo using the C57BL/KaLwRij mice injected i.v. with 10⁴ and/or 10⁵ 5T33 MM cells. Mice are treated with powdered regular chow, or 0.05% (W/W) M16αα in chow (about 50 mg/kg body weight/day), or M18γγ in chow (about 50 mg/kg body weight/day). At weekly intervals (in the kinetics study) and at the time of disease development, the mice are killed by CO₂ inhalation and the spleens, livers, thymi, and lymph nodes are excised and kept in PBS until processing for preparation of single-cell suspensions. Bone marrow from femora and tibiae are obtained by flushing PBS into the cavity of bones. Tumor load is quantified by determination of serum paraprotein by ELISA, or by capillary zone electrophoresis (Paragon). Plasmacytosis is determined by FACS analysis.

### Example 4

### Suppression of DSS-Induced Colitis by MEDICA Drugs

The involvement of MEDICA drugs in modulation of an immune response as also demonstrated using models of proliferative disorders, led the inventors to investigate the possible effect of MEDICA drugs on inflammatory disorders, such as IBD (Inflammatory Bowl Diseases), for example, Ulcerative Colitis and Crohn's Disease. Therefore, the DSS-induced colitis in mice has been used as model system for human ulcerative colitis.

Eight weeks old Balb/C male mice weighing 25 gr were raised under normal conditions with free access to water and rodent laboratory chow. Colitis was induced by adding 5% DSS (Dextran Sodium Sulfate, m.w 36,000-50,000) to their drinking water for seven days. M16αα dissolved in 1% CMC (Carboxy Methyl Cellulose) was administered daily by gavage (60 mg/kg/day, for 5 days followed by 30 mg/Kg/day for the last 4 days), starting two days prior to and through the seven days of DSS administration. Control mice were gavaged with the 1% CMC vehicle.

Upon sacrifice (at day 9), the entire colon was resected, weighed and its length measured. The disease activity index (DAI) was defined by the appearance of Diarrhea, Internal bleeding and External bleeding, as indicated in Experimental procedures. Colon samples were stored in buffered formalin, embedded in paraffin, sectioned, stained by H&E and analyzed histpathologically. Pathological score was defined by the Extent of colitis, Inflammation grade, Damage/Necrosis and Re-epithelializaion. Protein samples prepared from intestine frozen sections were analyzed by Western blot for STAT3, gp130 and Inhibitory kappaB (IkB) expression.

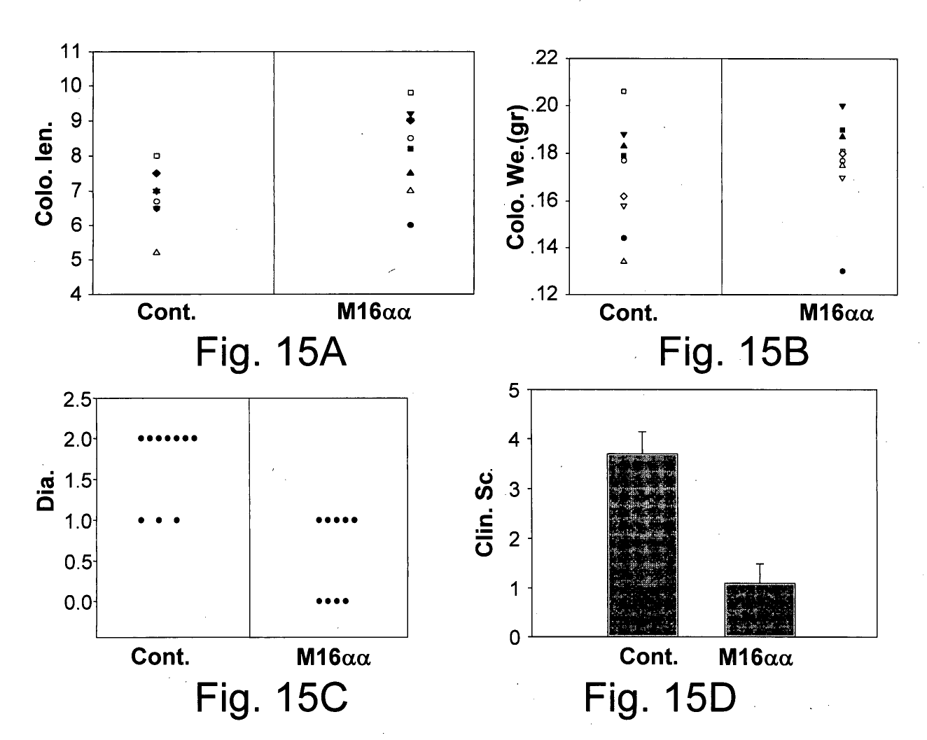

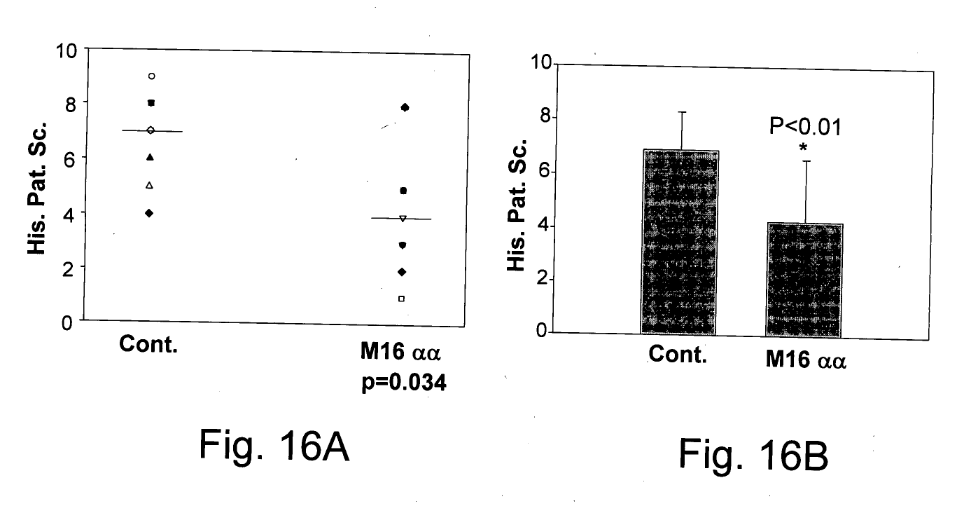

As shown by FIG. 15, treatment with MEDICA drugs clearly indicates alleviation in disease state. More specifically, the MEDICA treated animals showed improvement in colon length (FIG. 15A) and weight (FIG. 15B) as well as decrease in diarrhea (FIG. 15C) and clear reduction in clinical score (FIG. 15D). Similar improvement is also demonstrated by the histopathological score shown in FIG. 16A and the histogram of FIG. 16B.

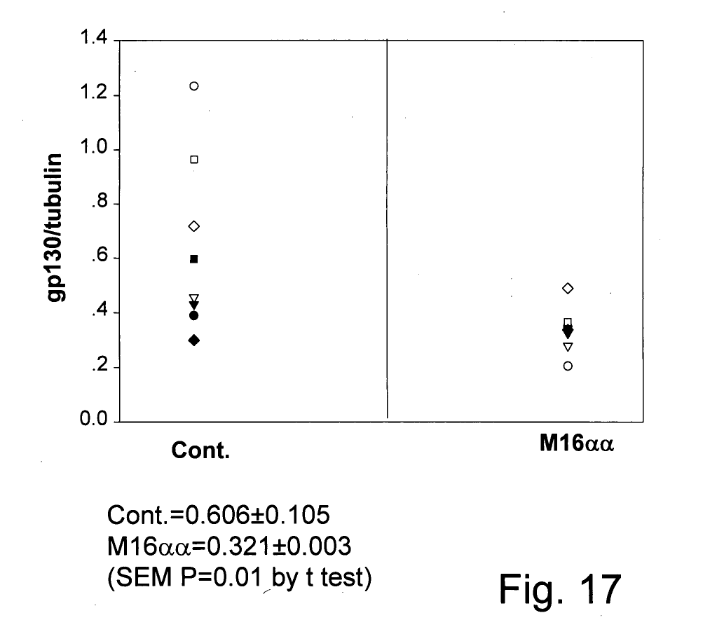

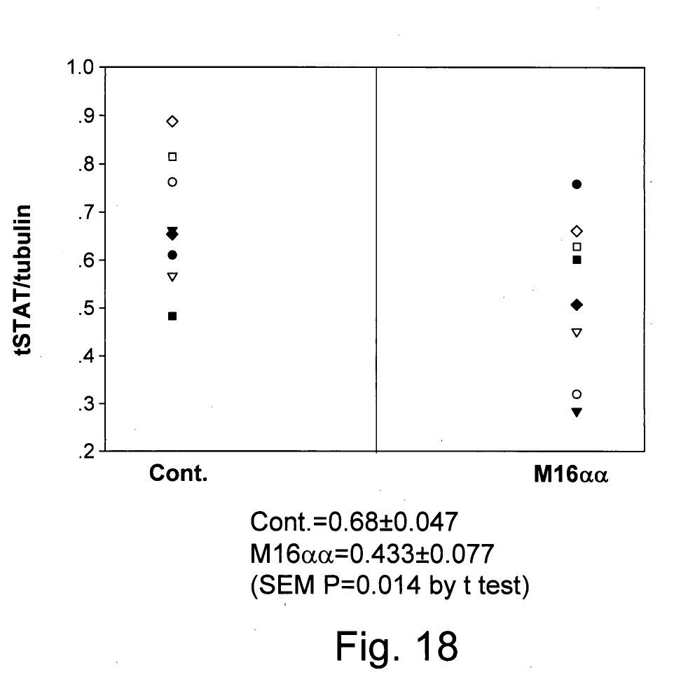

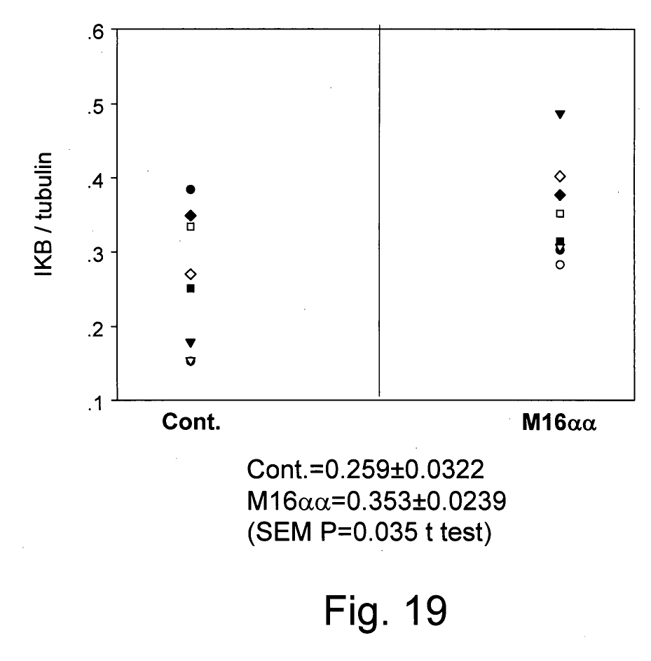

Analysis of inflammation markers similarly showed clear decrease in gp130 and STAT expression (FIGS. 17 and 20, respectively) and an increase in the expression of IkB (FIG. 19), in animals treated with MEDICA drugs. These results clearly demonstrate the anti-inflammatory effect of these drugs and the feasibility of uses thereof in the treatment of immune-related disorders.

### Example 5

### MEDICA Drugs in the Treatment and Prevention of Experimental Autoimmune Encephalomyelitis (EAE) in Mice

To further investigate the effect of the MEDICA drugs on immune-related disorders, the EAE as a model for Multiple Sclerosis (MS) has been next used. EAE was induced in 8-week-old female C57BL/6 mice by injecting myelin oligodendrocyte glycoprotein 35-55 peptide (MOG35-55) in the first and seventh days of the experiment. Mice were further inoculated with pertussis toxin in the first and second day of experiment as indicated in Experimental procedures. Four groups of mice (containing ten mice each) were evaluated for clinical score as detailed in experimental procedures. The first group (A), examining the potential preventive and protective effect of MEDICA drugs, received daily treatment with M16≢α (30 mg/kg BW) initiated three days prior to MOG injection and terminated on day 37, and followed by treatment with vehicle from day 37 to day 52. Mice of the second group (B) were treated daily with M16αα (30 mg/kg BW) starting on the seventh day following MOG injection, throughout the end of the experiment at day fifty two. The third group (C), received daily treatment with M16αα, (30 mg/kg BW) initiated on the first day throughout the end of the experiment at day fifty two.

The control group (D) was treated with vehicle (1% CMC) the first day throughout the end of the experiment at day fifty two.

As shown by FIG. 20, MOG challenge resulted in progressive EAE fully established on day 24, with an average maximal clinical score of 4.3. Treatment with M16αα initiated on the seventh day (group B) resulted in significantly delaying EAE development. Fully established EAE was delayed to day 40 with an average maximal clinical score of 4.3, Treatment with M16αα initiated on the first day (appearance of clinical signs in day twelve, group C), resulted in significantly delaying EAE development. Fully established EAE was delayed to day 40 with an average maximal clinical score of 3.8. Treatment of the Preventive Group A, initiated three days prior to MOG challenge, resulted in significantly delaying and abrogating EAE development, with an average maximal clinical score of 1.3. It should be noted that the EAE development in the “Preventive Group” was still abrogated upon termination of treatment, approaching an average maximal clinical score of 2.0 as compared with an average maximal clinical score of 4.3 for the untreated control group D.

These results clearly demonstrate the feasibility of using MEDICA drugs for the treatment and prevention of MS.

To further investigate the preventive potential of MEDICA drugs, and specifically of M16αα on EAE model, the effect of these drugs on different pathways involving activation of the immune system have been next examined. Therefore, female mice were treated with M16αα (60 mg/kg/day) or vehicle (1% CMC, the control group) for 12 days by gavage. On the third day EAE has been induced by injection of the MOG35-55 peptide. Nine days following MOG35-55 peptide immunization, mice were anesthesized and sacrificed. Spleenocytes were prepared and were stimulated in vitro with either MOG (100 μg/ml) or LPS (50 μg/ml) and the activation of STAT3 was analyzed by Western blot using anti p-STAT3 (Y705) and anti GP130 antibodies. Liver was immediately frozen in liquid nitrogen and kept at −70 until used for protein and RNA preparation. Total RNA was prepared and analyzed by real time PCR for SAP, SAA, α-fibrinogen and IL-6 gene expression.

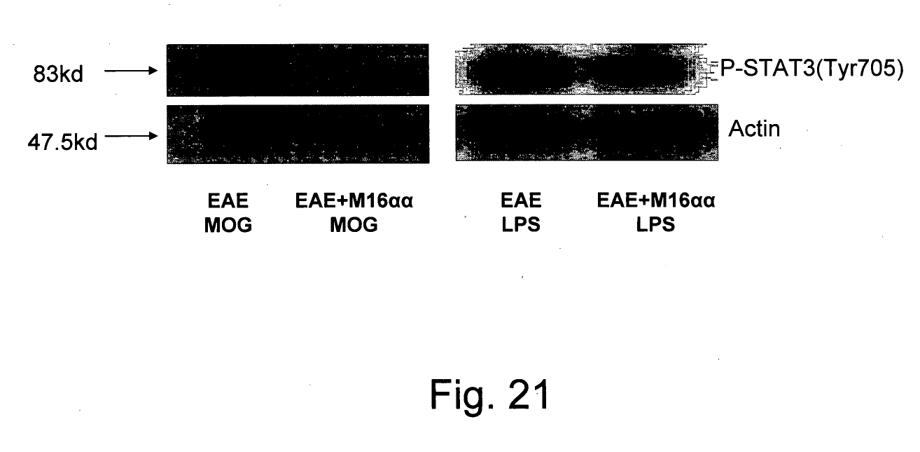

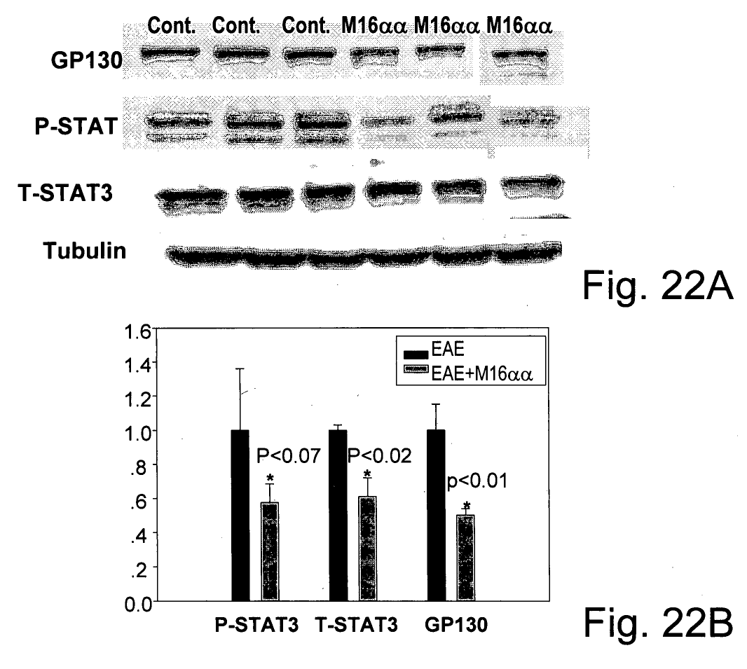

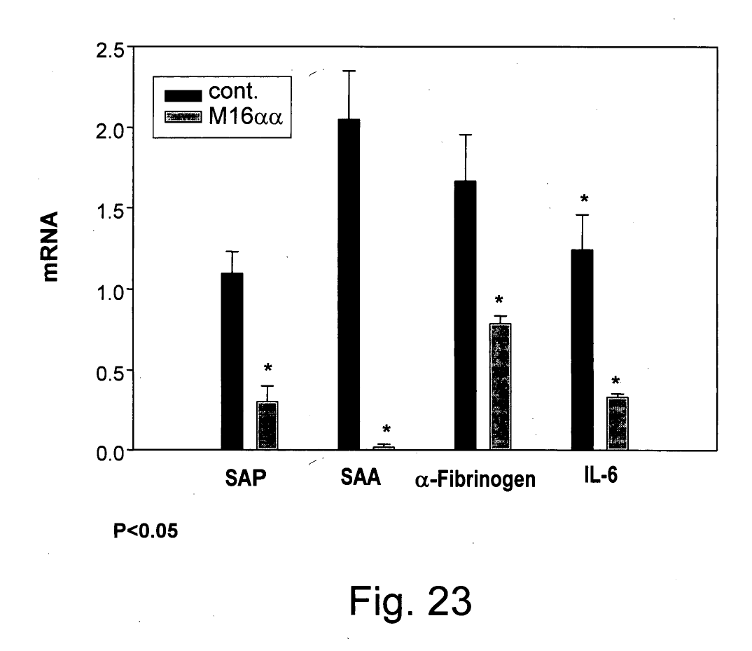

As shown by FIG. 21, only the MOG-induced, but not the LPS-induced STAT3 is suppressed in spleen cells from M16αα-treated EAE mice, indicating the specificity of this effect for the MOG antigen only. Similar results were also found using the GP130 antibodies, as demonstrated by the Western blot of FIG. 22A and the summarizing histogram of FIG. 22B. The effect of M16αα on the expression of liver Acute Phase Proteins (APP) is demonstrated by FIG. 23. As clearly shown, liver APP and IL-6 gene expression is robustly suppressed in M16αα-treated EAE mice, illustrating the anti-inflammatory clear effect of M16αα.

### Example 6

MEDICA Drugs for the Treatment of Atherosclerosis

The anti-inflammatory effect of the MEDICA drugs has been further examined for atherosclerosis using the CRP transgenic mice model. CRP is a risk factor and possible inflammatory causal agent, CRP directly acts as a pro-inflammatory stimulus inducing expression of intercellular adhesion molecule-1 (ICAM-1), vascular cell adhesion molecule-1 (VCAM-1), and monocytes chemotactic protein-1 (MCP-1) by human endothelial cells. Furthermore, CRP may induce interleukin-1β and TNFα, release by monocytes.

Unlike human CRP, mouse CRP is not an acute-phase reactant, and is synthesized hi trace amounts only. However, male hCRP transgene mice (hCRPtg), having the human CRP gene flanked by most of human CRP promoter integrated in their genome, constitutively produce human CRP, with serum levels ranging between 10 and 20 μg/mL. CRP levels in these transgenic mice are, comparable to or surpass those considered to indicate high risk in humans. Hence, hCRPtg have been used by the inventors as an animal model for studying the biological activities of human CRP in vivo, as well as studying the regulation of hCRP expression in response to MEDICA analogs.

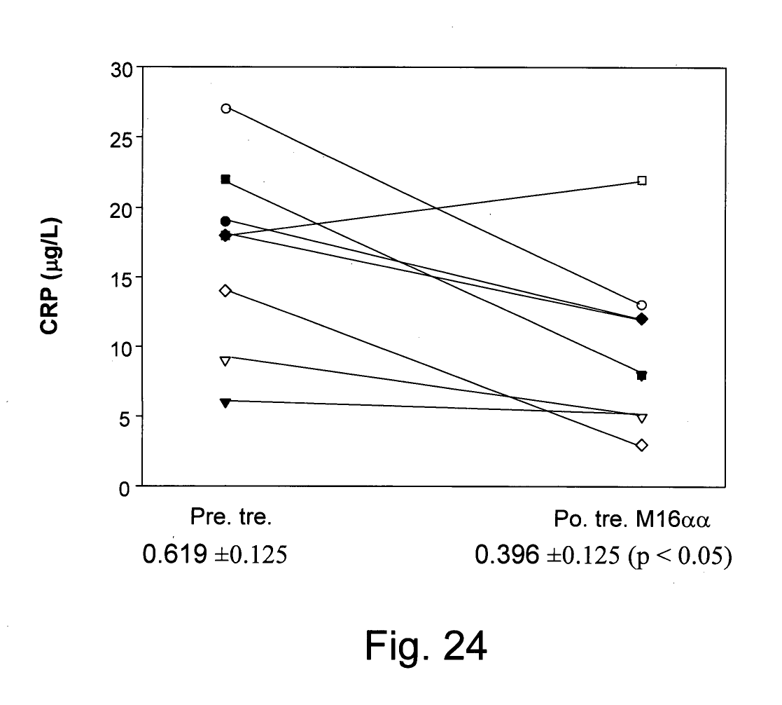

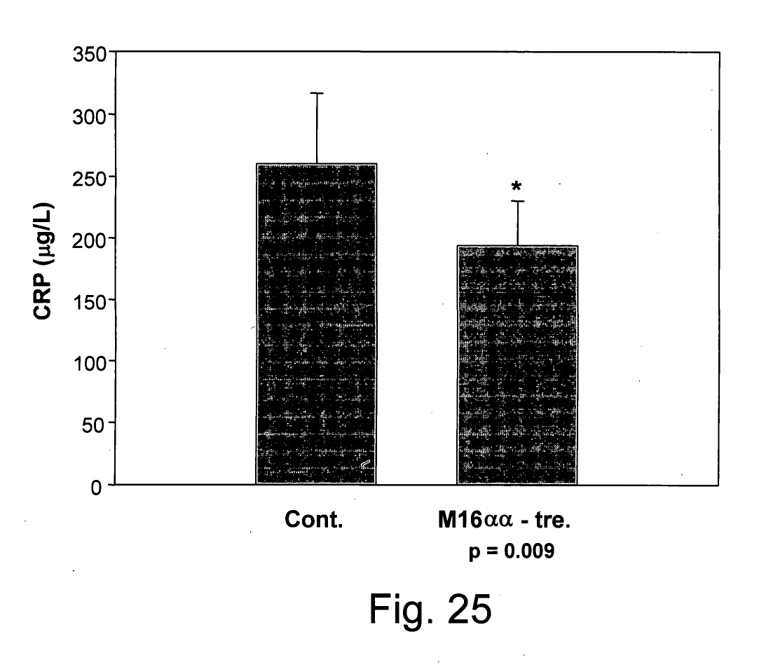

Therefore, hCRPtg mice were treated for ten days with M16αα (80 mg/kg/d) dissolved in 1% CMC and administered by gavage. Plasma CRP was determined by ELISA at the beginning and at the end of treatment. As shown by FIG. 24, treatment with M16αα resulted in a significant decrease in the basal levels of human CRP (0.619 compares to 0.396). The inventors further examined the effect of the MEDICA drugs on LPS-induced CRP. Therefore, CRP expression was analyzed in hCRPtg mice treated with M16αα 0.06% (W/W) (about 60 mg/kg body weight/day) for fourteen days and challenged with LPS (25 μg/mice) for the last 18 h. As illustrated by the histogram of FIG. 25, treatment with M16αα decreases LPS-induced levels of human CRP. These results demonstrate the feasibility of treating inflammatory disorders such as atherosclerosis using the MEDICA analogs of the invention.

1-29. (canceled)

30. A method for the treatment or prevention of an immune-related disorder comprising the step of administering to a subject in need thereof a therapeutically effective amount of at least one long-chain substituted amphipathic carboxylate or any salt, ester or amide thereof or any combination or mixture thereof and at least one additional therapeutic agent, or of any composition comprising the same, wherein said amphipathic carboxylate is any one of:
a compound denoted by formula (II): 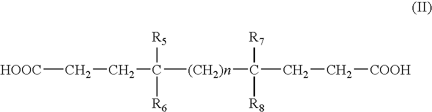
wherein n is an integer of from 2-14 and each of R₅, R₆, R₇ and R₈ represents lower alkyl;
a compound denoted by formula (III): 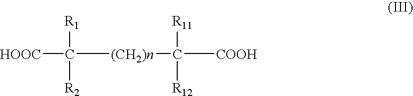
where n is an integer from 6 to 18 and each of R₁, R₂, R₁₁ and R₁₂ represents lower alkyl;
a compound denoted by formula (IV): 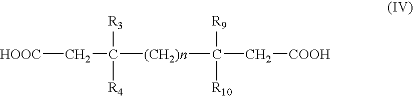
wherein n is an integer of from 4 to 16 and each of R₃, R₄, R₉ and R₁₀ represents lower alkyl; and said additional therapeutic agent is suitable for the treatment of immune and inflammatory conditions.

31. The method according to claim 30, wherein R₁ to R₁₂ each represents methyl.

32. The method according to claim 30, wherein said long-chain optionally substituted amphipathic dicarboxylic acid is a compound of Formula (II) being 4,4,15,15-tetramethyloctadecane-1,18-dioic acid (also referred to as M18γγ), or a compound of Formula (III) being 2,2,15,15-tetramethyl-hexadecane-1,16-dioic acid (also referred to as M16αα) or a compound of Formula (IV) being 3,3,14,14-tetramethyl-hexadecane-1,16-dioic acid (also referred to as M16ββ).

33. The method according to claim 32, wherein said immune-related disorder is any one of an inflammatory disease, an autoimmune disease and malignant and non-malignant proliferative disorders.

34. The method, according to claim 33, wherein said inflammatory disease is any one of multiple sclerosis, neurodegenerative disease, an atherosclerotic disease, an inflammatory bowel diseases and arthritis.

35. The method according to claim 34, wherein said atherosclerotic disease is any one of cardiovascular disease, cerebrovascular disease and peripheral vessel disease.

36. The method according to claim 33, wherein said malignant proliferative disorder is any one of carcinoma, myeloma, leukemia, lymphoma, sarcoma and melanoma.

37. The method according to claim 33, wherein said administration step comprises oral, intravenous, intramuscular, subcutaneous, intraperitoneal, parenteral, transdermal, intravaginal, intranasal, mucosal, sublingual, topical, rectal or subcutaneous administration, or any combination thereof.

38. The method according to claim 30, wherein said additional therapeutic agent is an immuno-modulatory agent.

39. The method according to claim 30, wherein said administration is periodical.

40. The method according to claim 30, wherein said administration is once, twice or three times daily.

41. An oral pharmaceutical composition for the treatment or prevention of an immune-related disorder comprising a therapeutically effective amount of at least one long-chain substituted amphipathic carboxylate or any salt, ester or amide amid thereof or any combination or mixture thereof, and optionally at least one additional therapeutic agent and optionally a pharmaceutically acceptable carrier, wherein said amphipathic carboxylate is any one of:
a compound denoted by formula (II): 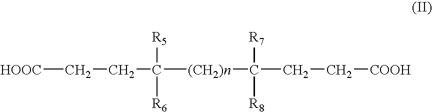
wherein n is an integer of from 2-14 and each of R₅, R₆, R₇ and R₈ represents lower alkyl;
a compound denoted by formula (III): 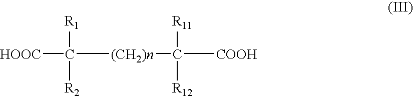
where n is an integer from 6 to 18 and each of R₁, R₂, R₁₁ and R₁₂ represents lower alkyl;
a compound denoted by formula (IV): 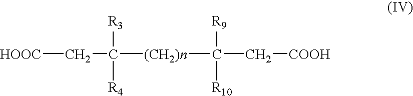
wherein n is an integer of from 4 to 16 and each of R₃, R₄, R₉ and R₁₀ represents lower alkyl; and said additional therapeutic agent is suitable for the treatment of immune and inflammatory conditions.

42. The composition according to claim 41, wherein R₁ to R₁₂ each represents methyl.

43. The composition according to claim 42, wherein said long-chain optionally substituted amphipathic dicarboxylic acid is a compound of Formula (II) being 4,4,15,15-tetramethyloctadecane-1,18-dioic acid (also referred to as M18γγ), or a compound of Formula (III) being 2,2,15,15-tetramethyl-hexadecane-1,16-dioic acid (also referred to as M16αα) or a compound of Formula (IV) being 3,3,14,14-tetramethyl-hexadecane-1,16-dioic acid (also referred to as M16ββ).

44. The composition according to claim 43, wherein said effective amount of any one of M16αα, MI6ββ or M18γγ administered daily, is from about 0.05 mg/kg to about 10 mg/kg of body weight.

45. The composition according to claim 41, wherein said additional therapeutic agent is an immuno-modulatory agent.

46. The composition according to claim 41, wherein said immune-related disorder is any one of an inflammatory disease, an autoimmune disease and malignant and non-malignant proliferative disorders.

47. A kit for achieving a therapeutic effect in the treatment or prevention of an immune-related disorder in a subject in need thereof comprising:
a. at least one long-chain substituted amphipathic carboxylate or any salt, ester or amid thereof or any combination or mixture thereof, or a pharmaceutically acceptable derivative thereof and a pharmaceutically acceptable carrier or diluent in a first unit dosage form, wherein said amphipathic carboxylate is as defined in claim 41;
b. at least one additional therapeutic agent, or a pharmaceutically acceptable derivative thereof and a pharmaceutically acceptable carrier or diluent in a second unit dosage form, wherein said additional therapeutic agent is suitable for the treatment of immune-related conditions; and
c. container means for containing said first and second dosage forms.
